(19) United States
(12) Patent Application Publication
(10) Pub. No.: US 2026/0085101 A1
(43) Pub. Date: Mar. 26, 2026

(54) NOVEL PLATELET DERIVED GROWTH FACTOR PEPTIDE MIMETIC

(71) Applicants: THE UNIVERSITY OF SYDNEY, New South Wales (AU); THE WESTMEAD INSTITUTE FOR MEDICAL RESEARCH, New South Wales (AU); WESTERN SYDNEY LOCAL HEALTH DISTRICT, New South Wales (AU); VICTOR CHANG CARDIAC RESEARCH INSTITUTE, New South Wales (AU)

(72) Inventors: James CHONG, New South Wales (AU); Emily McKINNA, New South Wales (AU); Richard PAYNE, New South Wales (AU); Daniel FORD, New South Wales (AU); Richard HARVEY, New South Wales (AU); Osvaldo CONTRERAS, New South Wales (AU)

(21) Appl. No.: 19/109,910

(22) PCT Filed: Sep. 8, 2023
(86) PCT No.: PCT/AU2023/050866

§ 371 Date: Mar. 7, 2025

(30) Foreign Application Priority Data
Sep. 9, 2022 (AU) 2022902612

(51) Int. Cl. C07K 14/49; A61K 38/00; A61K 47/54; A61K 47/64; A61P 9/00; A61P 9/10

(52) U.S. Cl. CPC C07K 14/49; A61K 47/542; A61K 47/643; A61P 9/00; A61P 9/10; A61K 38/00

(57) ABSTRACT

Disclosed herein are compounds of formula (I) wherein X, L1, and HSA conjugate are as defined herein, methods of preparation thereof, and methods of using thereof for improving cardiac repair, particularly post infarct.

### FIELD

The present invention relates to peptides useful for cardiac repair.

### BACKGROUND

There are currently no satisfactory treatment options available to reverse damage and restore cardiac function after large myocardial infarcts resulting in severe cardiac dysfunction. Although heart transplantation and ventricular assist devices are an option, limitations on supply and risks associated with these treatments mean that this is not a viable treatment option for most patients. For these reasons, considerable efforts towards research and development of cardiac regeneration are taking place. Several new strategies have been developed with promising pre-clinical results, however none have yet to be successfully translated into the clinic. These include gene therapy, cytokine therapies, cell-based therapies, and platelet-derived growth factor (PDGF) based therapies.

PDGF is one of many growth factors that regulate cell growth and division. The primary function of PDGF is in wound healing where it has a significant effect on mitogenesis, chemotaxis and angiogenesis. The mechanism for this is activation of the PDGF receptors (PDGFr) by autophosphorylation. The loop III region of the PDGF-B chain has been implicated in the mitogenic and chemotactic functions of PDGF as well as being significant in maintaining its receptor binding function. Loop III sits within the cysteine knot structure of PDGF and contains 12 amino acids, of which 5 play a significant role in receptor binding. When removed from or replaced within the chain, the result has been a loss in receptor binding and mitogenic function.

Pre-clinical studies in both murine and porcine models of myocardial infarction have shown that the AB heterodimer of recombinant PDGF increases cardiac repair by modulation of the cardiac scar and improved vascularity in the healing tissue.

Within the field of PDGF based therapies, researchers have also utilised the PDGF-B chain loop III to develop peptides. Specifically, Lin et al 2007 have shown that a PDGF peptide mimetic based on the PDGF-B chain loop III has in vitro effects that mimic native PDGF. This peptide was conjugated to a heparin binding domain (HBD).

However, PDGF are also well established as potent mitogens, with upregulation of PDGF resulting in increased proliferation. This upregulation has been associated with several disease states including fibrosis. Whilst being an integral component of wound healing, in the context of the heart, chronic fibrosis results in ventricular dysfunction which can result in the subsequent development of heart failure.

Fibrogenesis can occur as a result of increased proliferation of fibroblasts, often activated in wound healing by several growth factors, including PDGF. Given that PDGFs have known effects on the cell cycle and subsequent roles in fibrogenesis, work in this field has avoided prolonging exposure of PDGF based therapies.

Thus, there is a need for improved treatment options to reverse damage and restore cardiac function after large myocardial infarcts resulting in severe cardiac dysfunction. PDGF-based therapies provide a potential approach, as long as proliferation and fibrosis caused by PDGF is limited.

### SUMMARY OF INVENTION

The present invention alleviates at least one shortcoming associated with current treatments for reversing damage and restoring cardiac function after large myocardial infarcts.

Without limitation, the present invention provides compounds for reversing damage and restoring cardiac function following myocardial infarcts, methods of preparing such compounds, and methods for the prevention and/or treatment of various conditions or diseases by administration of such compounds.

### BRIEF DESCRIPTION OF DRAWINGS

FIG. 1. General scheme for solid phase peptide synthesis of compounds of formula MI.

FIG. 2. Migration of C2C12 Mouse Fibroblast Cells Treated with PDGF and peptides JC5 and JC5a. C2C12 mouse fibroblast cells migrate across the scratch over 24 hours. Cells were treated with 10 ng/ml PDGF−AB (n=6), PDGF−BB (n=6), and 1 μg/ml of novel PDGF mimetic JC5 (n=6) and JC5a (n=3). PDGF−BB (p<0.0001) and novel mimetic peptides JC5 (P value <0.0001) and JC5a (P value <0.0001) both showed significantly increased migration from the no treatment control and AG1296, negative controls. There was also no statistically significant difference between the PDGF mimetic peptides and PDGF−AB and −BB positive controls. Error bars represent ±SD.

FIG. 3. Collagen Contraction of C2C12 Mouse Fibroblasts Treated with PDGF and peptides JC5 and JC5a. C2C12 mouse fibroblasts significantly increase collagen contraction following treatment with 10 ng/ml of PDGF−AB (p<0.0001), PDGF−BB (p>0.0001) and 1 μg/mL of novel PDGF Mimetic peptides, JC5 (p>0.0001) and JC5a (p=0.0120) when compared to the negative control, AG1296+PDGF−AB. PDGF−AB (p=0.0073), PDGF−BB (p=0.0126) and novel PDGF mimetic JC5 (p=0.0100) also significantly increased collagen contraction when compared to the no treatment control group. Error bars represent ±SD, n=8/4.

FIG. 4. Tube formation of Human Coronary Artery Endothelial Cells Treated with PDGF and PDGF Mimetic Peptide JC5. Human coronary artery endothelial cells treated with 50 ng/ml of PDGF−AB and PDGF−BB produced longer tubes than untreated cells and cells treated with the PDGF receptor inhibitor AG1296+PDGF−AB. Cells treated with 5 μg/ml of JC5 also resulted in increased tube length when compared to the no treatment and AG1296+PDGF−AB negative controls. Treatment with PDGF−AB, PDGF−BB and JC5 resulted in significantly increased tube length versus the no treatment control (AB− p=0.0062, BB− p=0.0013, JC5− p=0.0025) and AG1296+PDGF−AB negative control (AB− p=0.0068, BB− p=0.0014, JC5−p=0.0027). Error bars represent ±SD, n=3/group.

FIG. 5. Phosphorylation of ERK following treatment of C2C12 Cells with PDGF and JC5. Western blots for total (ERK) and phosphorylated ERK (pERK) at 42 and 44 kDa show an increase in phosphorylation following treatment with 10 ng/ml PDGF−BB and 1 μg/ml of JC5.

FIG. 6. Phosphorylation of ERK following treatment of C2C12 Cells with PDGF and JC5a. Western blots for total (ERK) and phosphorylated ERK (pERK) at 42 and 44 kDa show an increase in phosphorylation 10 minutes post treatment with 10 ng/ml PDGF−BB and 1 μg/ml of novel mimetic peptide JC5a. JC5a maintained this increased phosphorylation of ERK at 30 minutes post treatment.

FIG. 7. Annotated formula (I).

FIG. 8. Proliferation of C3H10T½ Cells Following Treatment with PDGF and Novel PDGF Mimetic Peptides. Cultured C3H10T½ mouse embryonic fibroblast cells were treated with 20 ng/ml recombinant PDGF−AB or PDGF−BB protein ligands, and 2 μg/ml of novel PDGF mimetics JC5 or JC5a for 24 hr, in the presence of 1% fetal bovine serum (FBS). Percent proliferating cell was determined after a 2 hr pulse with nucleotide analogue EdU at the end of the incubation period. PDGF−BB induced significantly greater proliferation when compared to the 1% FBS control. However, PDGF−AB, JC5 and JC5a did not significantly increase proliferation versus 1% FBS and were not significantly different to each other. Thus, unlike PDGF-BB, JC5 and JC5a do not show significant proliferative activity. Co-treatment of protein/peptides with 10 μM of PDGF receptor antagonist, AG1296, confirmed that PDGF-BB-stimulated proliferation occurred via the PDGF ligand/receptor pathway. AG1296 also significantly inhibited PDGF−AB and JC5 EdU uptake, hinting at a weak effect via PDGF receptors for these treatments. Error bars represent ±SD, n=3. Statistical analysis was using One-Way Anova: * p<0.05; ** p<0.01; *** p<0.001; **** p<0.0001.

FIG. 9: Improved Cardiac function at Day 28 Post Myocardial Infarction. A) C57BL6/J mice treated with PDGF−AB (p=0.0073) and novel mimetic peptide JC5 (p=0.0129) had a significantly increased ejection fraction at day 28 versus the PBS vehicle control group. B) Mice treated with positive control PDGF−AB (p=0.0003) and novel mimetic peptide JC5 (p=0.0032) resulted in significantly decreased end systolic volume versus mice treated with the vehicle control PBS. C) A decrease in end diastolic volumes was also observed in PDGF−AB (p=0.0016) and JC5 (p=0.0185) treated mice versus PBS. D) Both PDGF−AB (p=0.0393) and JC5 (p=0.0409) treated mice had a significant day 2-day 28 Δ ejection fraction versus the PBS vehicle control. Novel mimetic peptide JC5a (p=0.0655) also had an increased day 2-day 28 Δ ejection fraction (p=0.0082) when compared to PBS. E) Timeline of mean EF per treatment group. Baseline indicates the pre-infarct EF per group. Day 2 and day 28 indicates days post infarct. A-C) Error bars represent ±SD. D) Data presented as median ±minimum and maximum values. One way ANOVA comparing all groups to the PBS vehicle control group (n=10/11).

FIG. 10: Inter-observer variability of Day 28 Ejection Fraction. Inter-observer variability at day 28 was within normal limits for rodent echocardiography with a bias of 14.06 (±7.346). Error bars (dotted lines) represent 95% limits of agreement.

FIG. 11: Decreased Heart Weight/Tibia Length in Mice Treated with JC5. Heart weight normalised by tibia length was significantly decreased in C57BL6/J mice treated with positive control PDGF−AB (p=0.0066) (n=10) and novel mimetic peptide JC5 (p=0.0366) (n=11) when compared to the vehicle control PBS (n=10). Error bars represent ±SD.

### DEFINITIONS

As used in this application, the singular form “a”, “an” and “the” include plural references unless the context clearly dictates otherwise.

As used herein, the term “comprising” means “including.” Variations of the word “comprising”, such as “comprise” and “comprises,” have correspondingly varied meanings.

It will be understood that use the term “about” herein in reference to a recited numerical value includes the recited numerical value and numerical values within plus or minus ten percent of the recited value.

It will be understood that use of the term “between” herein when referring to a range of numerical values encompasses the numerical values at each endpoint of the range. For example, a temperature of between 80° C. and 150° C. is inclusive of a temperature of 80° C. and a temperature 150° C.

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

### DESCRIPTION OF EMBODIMENTS

The present inventors have surprisingly found that linkage of a dimer of the loop III region of the PDGF-B chain of PDGF to a fatty acid conjugate of human serum albumin (HSA) results in a compound of formula (I), mimics the useful properties of PDGF for cardiac repair while avoiding the undesirable side effects of PDGF-based therapies such as increased cell proliferation and fibrosis.

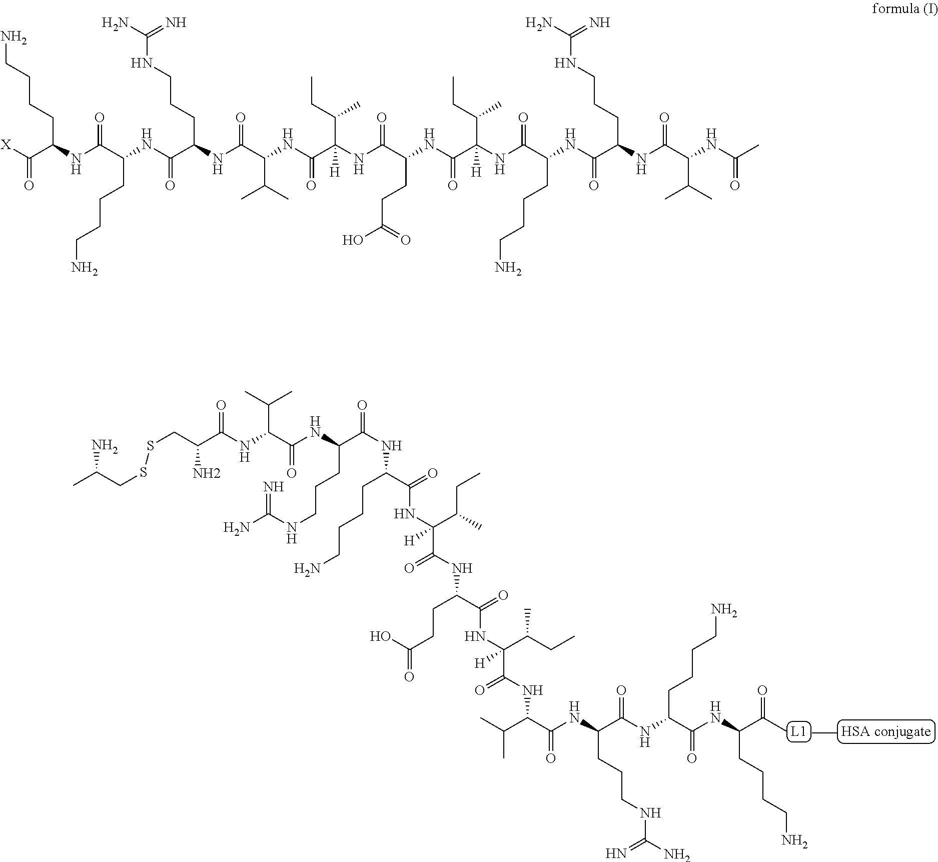

wherein X═NH₂ or -L2-[HSA conjugate], wherein

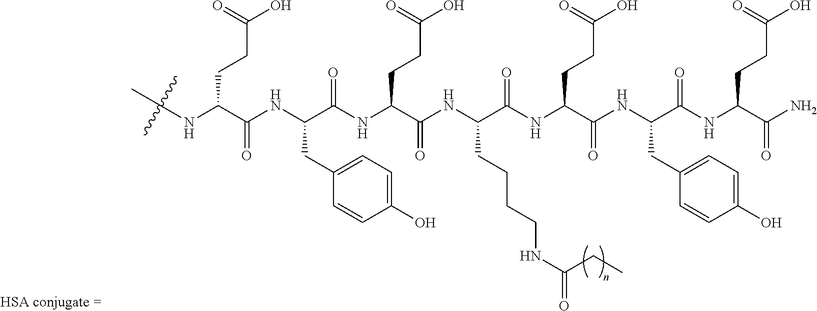

and n, L1 and L2 are as described herein.

The HSA conjugate of the invention is designed to extend the half-life of therapeutic peptides by binding them to the abundant carrier protein albumin, prolonging the time to clearance via renal filtration. However, this particular HSA conjugate results in a shorter half-life extension than a full albumin fusion protein due to its inability to exploit the FcRn recycling pathway. It is anticipated that this will extend the half-life of the dimer of the loop III region of the PDGF-B chain by up to 25-fold, whilst not extending it beyond several days.

The inventors theorise that this conjugation allows for improved clinical use and provides more time for the mechanism of action of the peptide to take place, whilst ensuring it has been excreted prior to the onset of potentially problematic side effects such as fibrogenesis. Thus the compounds of the invention unexpectedly provide activity which mimics the useful properties of PDGF while avoiding excessive and harmful fibrogenesis.

### Compounds

In one aspect of the invention, there is provided a compound of formula (I) or salt thereof:

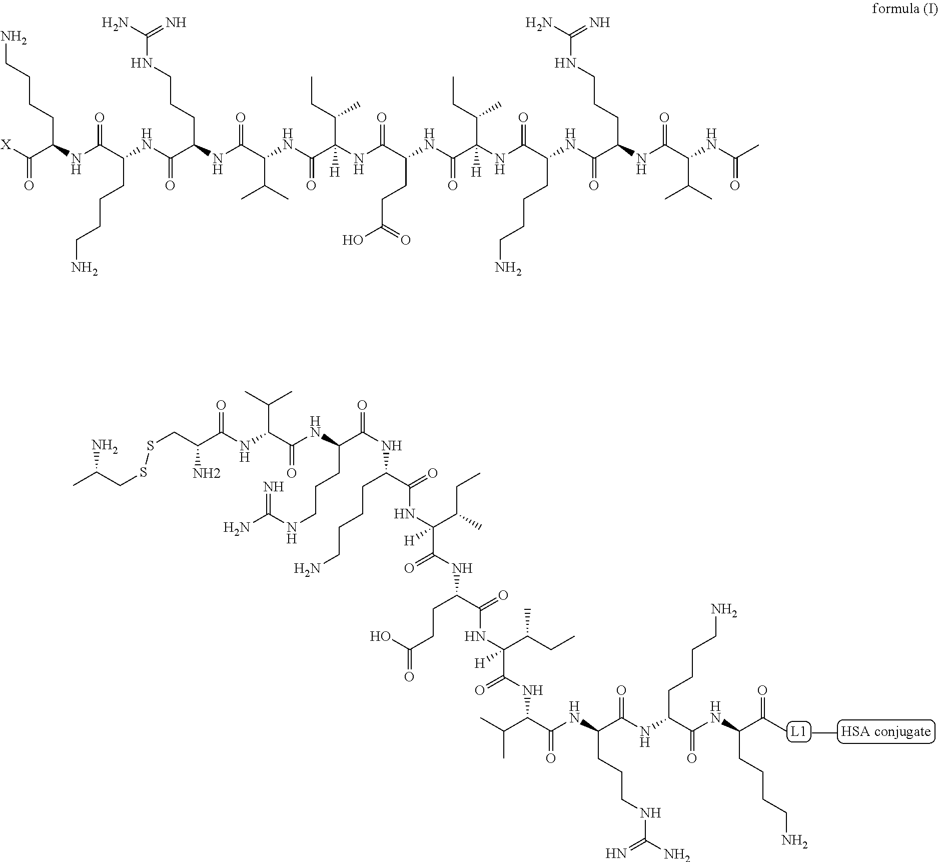

wherein X═NH₂ or -L2-[HSA conjugate], wherein

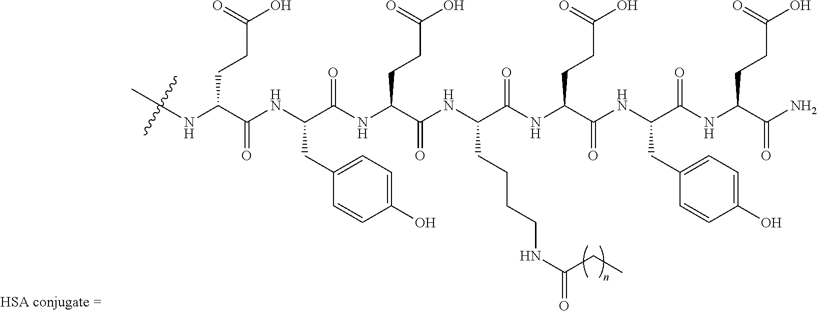

wherein L1 and L2 are independently selected from the group consisting of —HN(C₂-C₆₀ alkyl)C(O)—, —HN(C₂-C₆₀ alkaryl)C(O)—, —HN(C₂-C₆₀ alkenyl)C(O)—, —HN(CH₂)ᵣ(CH₂CH₂O)ₒ(CH₂)ᵣC(O)—, —HN(CH₂)ᵣ(O(CH₂)ₘC(O))ₒ(CH₂)ᵣC(O)—, —HN(CH₂)ᵣ ((NH(CH₂)ₘC(O))ₒ)(CH₂)ᵣC(O)—, and

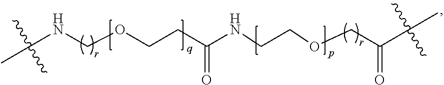

wherein n=1 to 20, o=1 to 60, r=2 to 20, m=1 to 60, p=1 to 60,and q=1 to 60.

L1 and L2 may be the same or L1 and L2 may be different.

In one embodiment, L1 and L2 are independently selected from the group consisting of —HIN(C₂-C₂₀ alkyl)C(O)—, —HIN(C₂-C₂₀ alkaryl)C(O)—, —HIN(C₂-C₂₀ alkenyl)C(O)—, —HN(CH₂)ᵣ(CH₂CH₂O)ₒ(CH₂)ᵣC(O)—, —HN(CH₂)ᵣ(O(CH₂)ₘC(O))ₒ(CH₂)ᵣC(O)—, —HN(CH₂)ᵣ ((NH(CH₂)ₘC(O))ₒ)(CH₂)ᵣC(O)—, and

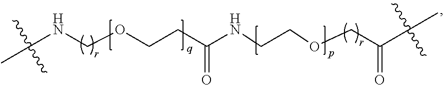

wherein n=1 to 20, o=1 to 20, r=2 to 20, m=1 to 20, p=1 to 20, and q=1 to 20.

In each incidence of HSA conjugate in formula (I), n may be the same or n may be different.

—HN(C₂-C₆₀ alkyl)C(O)— means a branched or linear alkyl chain having 2 to 60 carbons, such as —HN—(CH₂)ₐ-C(O)—, wherein a=2 to 60. In some embodiments, a=2 to 20. —HN(C₂-C₆₀ alkaryl)C(O)— means a linear chain containing one or more aryl groups, such as —HN—[(CH₂)_{b}—C₆H₄—(CH₂)_{c}]_{d}—C(O)—, wherein b=0 to 60, c=0 to 60, and d=1 to 60. In some embodiments, b=0 to 20, c=0 to 20, and d=1 to 20. —HN(C₂-C₆₀ alkenyl)C(O)— means a linear chain containing one or more alkene groups, such as —HN—[(CH₂)_{b}—CH═CH—(CH₂)_{c}]_{d}—C(O)—, wherein b=0 to 60, c=0 to 60, and d=1 to 60. In some embodiments, b=0 to 20, c=0 to 20, and d=1 to 20.

In an embodiment, n=15.

In an embodiment, r=2.

In some embodiments, L1 and L2 may be

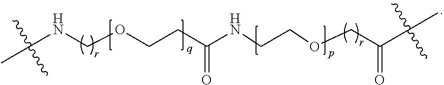

In particular, L1 and L2 may be

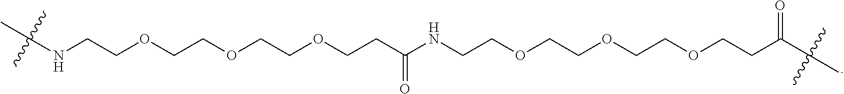 [SMILES: *NCCOCCOCCOCCC(=O)NCCOCCOCCOCCC(*)=O]

In one embodiment where X is NH₂, the dimer of the loop III region of the PDGF-B chain is linked to a single HSA conjugate. In this case, the compound of formula (I) may be Compound Ia:

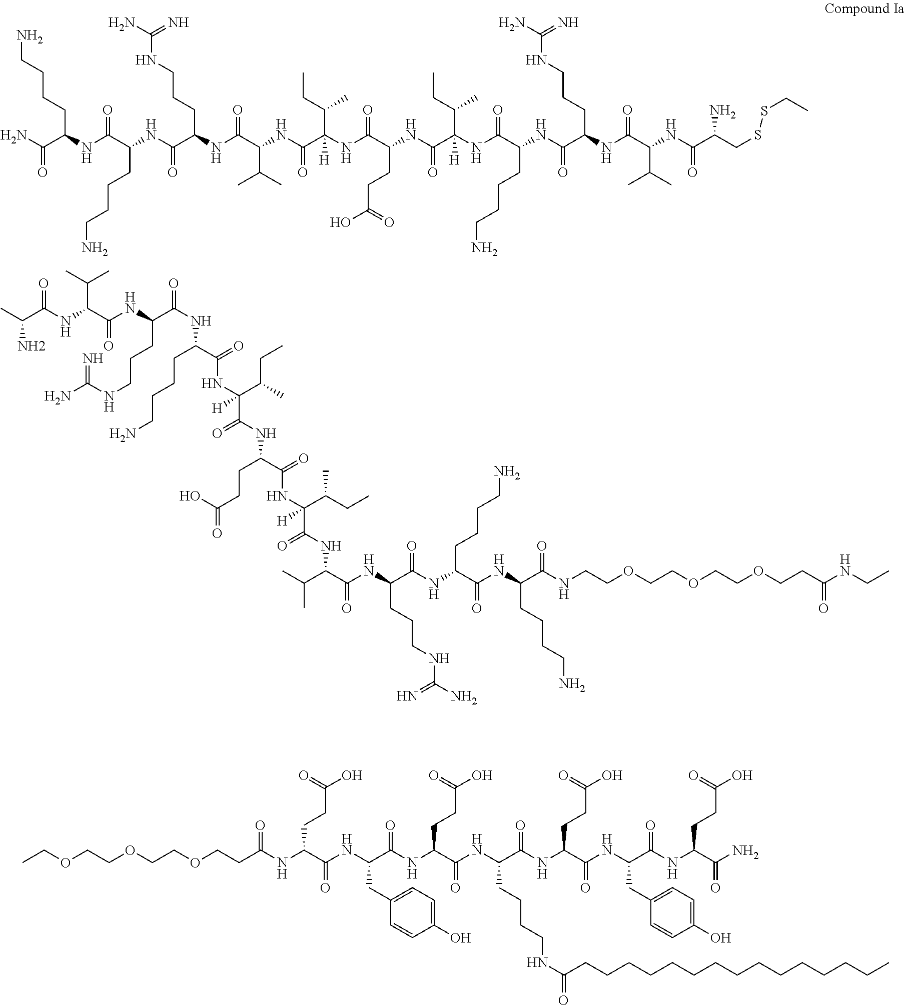

In another embodiment where X is -L2-[HSA conjugate], the dimer of the loop III region of the PDGF-B chain is linked to two HSA conjugates. In this case, the compound of formula (I) may be Compound Ib:

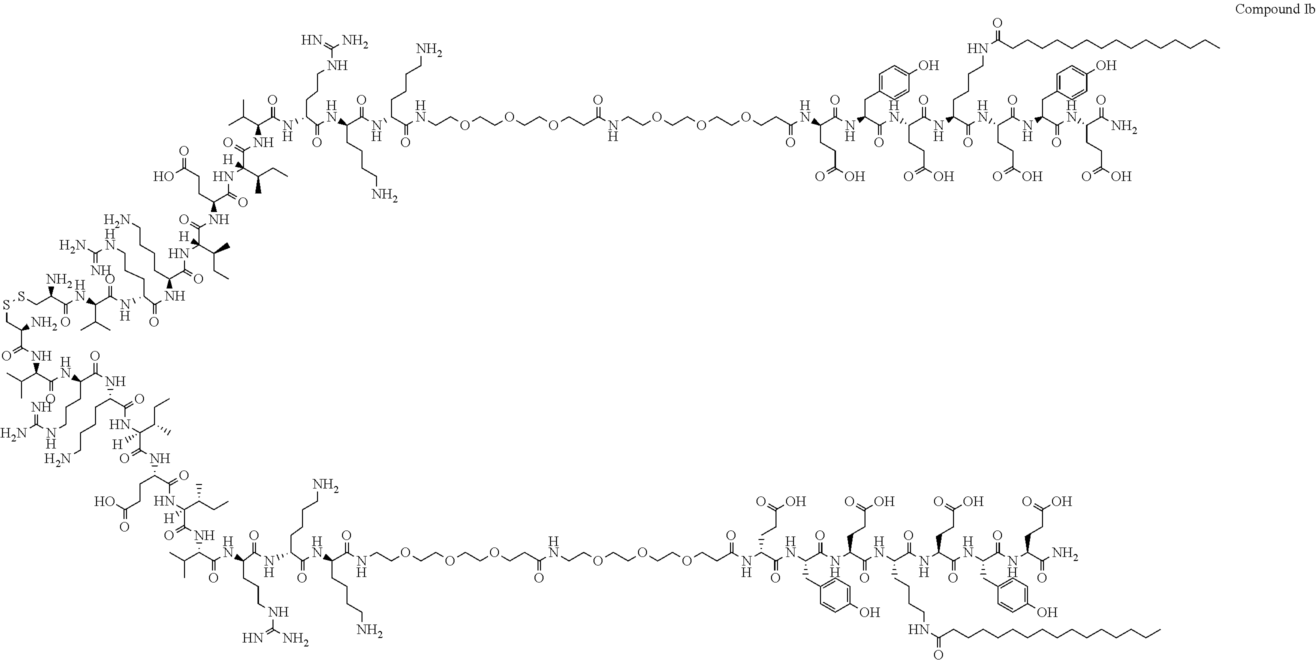

In one embodiment the compounds of formula (I) exist as their free base. In another embodiment of the compounds of formula (I) exist as pharmaceutically acceptable salts. The phrase “pharmaceutically acceptable salt” refers to any salt preparation that is appropriate for use in a pharmaceutical application. By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are described, for example in J. Pharmaceutical Sciences, 1977, 66:1-19. In one embodiment the pharmaceutically acceptable salt is a hydrochloride salt. The compounds of formula (I) may also exist as any suitable salt, for example a trifluoroacetic acid salt or a formic acid salt.

### Methods of Preparation

In another aspect, there is provided a method of preparing a compound of formula (I) or salt thereof.

First, the method of preparing a compound of formula (I) comprises preparing a PDGF Loop III monomer (Compound II) of the structure:

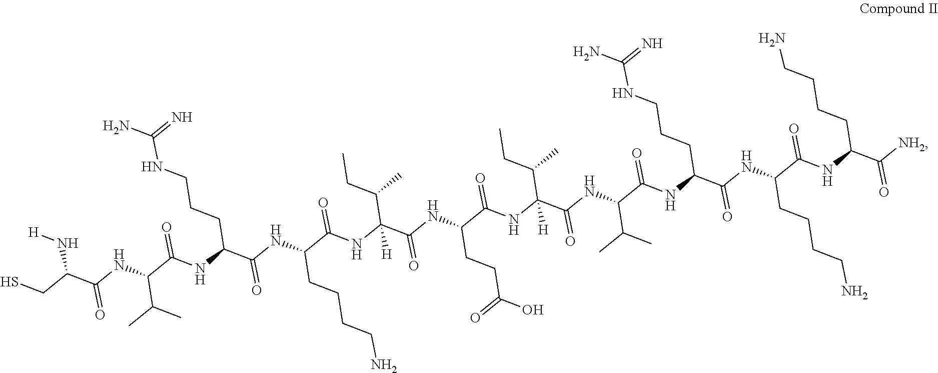 [SMILES: CC[C@H](C)[C@H](NC(=O)[C@H](CCCCN)NC(=O)[C@H](CCCNC(=N)N)NC(=O)[C@@H](NC(=O)[C@@H](N)CS)C(C)C)C(=O)N[C@@H](CCC(=O)O)C(=O)N[C@H](C(=O)N[C@H](C(=O)N[C@@H](CCCNC(=N)N)C(=O)N[C@@H](CCCCN)C(=O)N[C@@H](CCCCN)C(N)=O)C(C)C)[C@@H](C)CC]

The PDGF Loop III monomer is conveniently prepared by solid-phase peptide synthesis, for example Fmoc solid-phase peptide synthesis. Solid-phase peptide synthesis is a well-known, standard technique for preparing polypeptides. Typically and in general, the protected C-terminal amino acid residue is provided, covalently linked to polystyrene resin by functional group that may be cleaved under suitable conditions. The terminal residue is deprotected and the subsequent amino acid is coupled by an amide coupling to form a dipeptide linked to the resin. This cycle is repeated until the complete polypeptide is obtained, covalently linked to the resin. The polypeptide may then cleaved from the resin and deprotected.

Second, the PDGF Loop III monomer is joined to the HSA conjugate and a linker to form a linked PDGF Loop III HSA monomer (formula (II)):

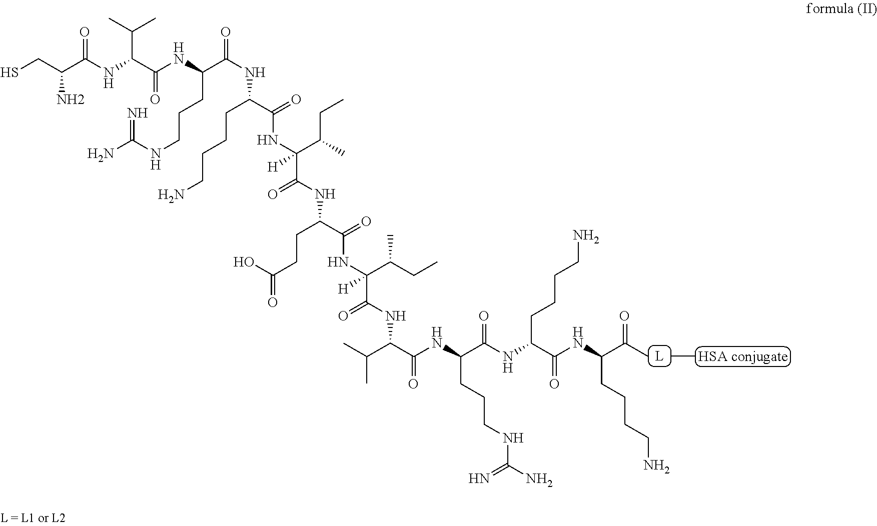

This step may also be carried out using solid-phase peptide synthesis, particularly as the linker and HSA conjugate are also connected by amide bonds. That is, the PDGF Loop III monomer is prepared by solid-phase peptide synthesis, followed by coupling of the linked HSA conjugate. The linked PDGF Loop III HSA monomer is then cleaved from the resin.

Third, the linked PDGF Loop III HSA monomer of formula (II) may be joined to the PDGF Loop III monomer (Compound II) by disulfide coupling. This forms, for example, Compound Ia. Alternatively, two linked PDGF Loop III HSA monomers of formula (II) may be joined to each other by disulfide coupling. This forms, for example, Compound Ib. The disulfide coupling may be carried out under any suitable conditions, such as in saturated aqueous NH₄⁺CO₃⁻.

In alternative embodiments, compounds of formula (I) or salts thereof may be prepared using native chemical ligation, or a combination of solid-phase peptide synthesis and native chemical ligation.

### Compositions

In another aspect of the invention, there is provided a composition comprising a compound of formula (I) and a pharmaceutically acceptable excipient, carrier and/or diluent.

Compositions of the invention may contain the compound of formula (I) as the sole therapeutic agent. Alternatively, compositions of the invention may contain the compound of formula (I) in combination with an additional therapeutic agent.

Compositions of the invention may be formulated for oral administration or for parenteral administration. Parenteral administration of compounds of the invention may be, for example, by intravenous injection, intra-arterial injection, intra-coronary arterial injection, intra-coronary venous injection, subcutaneous injection, gastric auto-injector, an implanted minipump, transdermal patch, an implantable system allowing administration to local tissues by use of bioengineered constructs, a gastrointestinal mucoadhesive patch system, or a microneedle system. Compositions of the invention may be formulated in any suitable manner to deliver an effective amount of the compound of formula (I), for example in a nanoparticle formulation, polymer matrix formulation, or lipid formulation.

### Methods and Uses

Compounds of formula (I) are useful in the methods and uses described below by mimicking functions of PDGF such as chemotaxis, collagen contraction, vascular tube formation and activation of the PDGF receptors via phosphorylation of Akt and ErK.

In embodiments of the below method and uses, the subject may have experienced myocardial infarct, or alternatively, the subject may not have experienced myocardial infarct. In embodiments of the below methods and uses, the chronic heart failure may be ischemic heart failure, or may be non-ischemic heart failure. In some embodiments, where the subject is suffering from non-ischemic heart failure, the subject may not have experienced myocardial infarct.

In one aspect, there is provided a method of reversing cardiac damage caused by myocardial infarct in a subject in need thereof, the method comprising administering the compound of formula (I) or salt thereof, or the pharmaceutical composition of the invention to the subject.

In another aspect, there is provided a method of reversing cardiac damage in a subject suffering from chronic heart failure, the method comprising administering the compound of formula (I) or salt thereof, or the pharmaceutical composition of the invention to the subject.

In another aspect, there is provided a method of treating cardiac damage caused by myocardial infarct in a subject in need thereof, the method comprising administering the compound of formula (I) or salt thereof, or the pharmaceutical composition of the invention to the subject.

In another aspect, there is provided a method of treating cardiac damage in a subject suffering from chronic heart failure, the method comprising administering the compound of formula (I) or salt thereof, or the pharmaceutical composition of the invention to the subject.

In another aspect, there is provided a method of restoring or improving cardiac function following myocardial infarct in a subject in need thereof, the method comprising administering the compound of formula (I) or salt thereof, or the pharmaceutical composition of the invention to the subject.

In another aspect, there is provided a method of restoring or improving cardiac function in a subject suffering from chronic heart failure, the method comprising administering the compound of formula (I) or salt thereof, or the pharmaceutical composition of the invention to the subject.

In another aspect, there is provided a method of enhancing cardiac repair following myocardial infarct in a subject in need thereof, the method comprising administering the compound of formula (I) or salt thereof, or the pharmaceutical composition of the invention to the subject.

In another aspect, there is provided a method of enhancing cardiac repair in a subject suffering from chronic heart failure, the method comprising administering the compound of formula (I) or salt thereof, or the pharmaceutical composition of the invention to the subject.

In another aspect, there is provided a method of treatment of cardiac dysfunction following myocardial infarction in a subject in need thereof, the method comprising administering the compound of formula (I) or salt thereof, or the pharmaceutical composition of the invention to the subject.

In another aspect, there is provided a method of treatment of cardiac dysfunction in a subject suffering from chronic heart failure, the method comprising administering the compound of formula (I) or salt thereof, or the pharmaceutical composition of the invention to the subject.

In another aspect, there is provided a method of treatment of persistent angina following myocardial infarction in a subject in need thereof, the method comprising administering the compound of formula (I) or salt thereof, or the pharmaceutical composition of the invention to the subject.

In another aspect, there is provided a method of treatment of persistent angina in a subject suffering from chronic heart failure, the method comprising administering the compound of formula (I) or salt thereof, or the pharmaceutical composition of the invention to the subject.

In another aspect, there is provided a method of treatment of persistent angina in a subject in need thereof, the method comprising administering the compound of formula (I) or salt thereof, or the pharmaceutical composition of the invention to the subject.

In another aspect, there is provided a method of improving survival, decreasing arrhythmias, and/or increasing ventricular contraction and compliance following myocardial infarction in a subject in need thereof, the method comprising administering the compound of formula (I) or salt thereof, or the pharmaceutical composition of the invention to the subject.

In another aspect, there is provided a method of improving survival, decreasing arrhythmias, and/or increasing ventricular contraction and compliance in a subject suffering from chronic heart failure, the method comprising administering the compound of formula (I) or salt thereof, or the pharmaceutical composition of the invention to the subject.

In another aspect, there is provided a method of prolonging survival of a subject following myocardial infarction, the method comprising administering the compound of formula (I) or salt thereof, or the pharmaceutical composition of the invention to the subject.

In another aspect, there is provided a method of prolonging survival of a subject suffering from chronic heart failure, the method comprising administering the compound of formula (I) or salt thereof, or the pharmaceutical composition of the invention to the subject.

In another aspect, there is provided a method of preventing the development of severe heart failure in a subject following myocardial infarction, the method comprising administering the compound of formula (I) or salt thereof, or the pharmaceutical composition of the invention to the subject.

In another aspect, there is provided a method of preventing the development of severe heart failure in a subject suffering from chronic heart failure, the method comprising administering the compound of formula (I) or salt thereof, or the pharmaceutical composition of the invention to the subject.

In another aspect, there is provided a method of treatment of chronic heart failure in a subject in need thereof, the method comprising administering the compound of formula (I) or salt thereof, or the pharmaceutical composition of the invention to the subject.

In another aspect, there is provided the compound of formula (I) or salt thereof for use in reversing cardiac damage caused by myocardial infarct in a subject in need thereof.

In another aspect, there is provided the compound of formula (I) or salt thereof for use in reversing cardiac damage in a subject suffering from chronic heart failure in a subject in need thereof

In another aspect, there is provided the compound of formula (I) or salt thereof for use in treating cardiac damage caused by myocardial infarct in a subject in need thereof.

In another aspect, there is provided the compound of formula (I) or salt thereof for use in treating cardiac damage in a subject suffering from chronic heart failure.

In another aspect, there is provided the compound of formula (I) or salt thereof for use in restoring or improving cardiac function following myocardial infarct in a subject in need thereof.

In another aspect, there is provided the compound of formula (I) or salt thereof for use in restoring or improving cardiac function in a subject suffering from chronic heart failure.

In another aspect, there is provided the compound of formula (I) or salt thereof for use in enhancing cardiac repair following myocardial infarct in a subject in need thereof.

In another aspect, there is provided the compound of formula (I) or salt thereof for use in enhancing cardiac repair in a subject suffering from chronic heart failure.

In another aspect, there is provided the compound of formula (I) or salt thereof for use in treatment of cardiac dysfunction following myocardial infarction in a subject in need thereof.

In another aspect, there is provided the compound of formula (I) or salt thereof for use in treatment of cardiac dysfunction in a subject suffering from chronic heart failure.

In another aspect, there is provided the compound of formula (I) or salt thereof for use in treatment of persistent angina following myocardial infarction in a subject in need thereof.

In another aspect, there is provided the compound of formula (I) or salt thereof for use in treatment of persistent angina in a subject suffering from chronic heart failure.

In another aspect, there is provided the compound of formula (I) or salt thereof for use in treatment of persistent angina.

In another aspect, there is provided the compound of formula (I) or salt thereof for use in improving survival, decreasing arrhythmias, and/or increasing ventricular contraction and compliance following myocardial infarction in a subject in need thereof.

In another aspect, there is provided the compound of formula (I) or salt thereof for use in improving survival, decreasing arrhythmias, and/or increasing ventricular contraction and compliance in a subject suffering from chronic heart failure.

In another aspect, there is provided the compound of formula (I) or salt thereof for use in prolonging survival of a subject following myocardial infarction.

In another aspect, there is provided the compound of formula (I) or salt thereof for use in prolonging survival of a subject suffering from chronic heart failure.

In another aspect, there is provided the compound of formula (I) or salt thereof for use in preventing the development of severe heart failure in a subject following myocardial infarction.

In another aspect, there is provided the compound of formula (I) or salt thereof for use in preventing the development of severe heart failure in a subject suffering from chronic heart failure.

In another aspect, there is provided the compound of formula (I) or salt thereof for use in treatment of chronic heart failure in a subject in need thereof.

In another aspect, there is provided use of the compound of formula (I) or salt thereof in the manufacture of a medicament for reversing cardiac damage caused by myocardial infarct in a subject in need thereof.

In another aspect, there is provided use of the compound of formula (I) or salt thereof in the manufacture of a medicament for reversing cardiac damage in a subject suffering from chronic heart failure.

In another aspect, there is provided use of the compound of formula (I) or salt thereof in the manufacture of a medicament for treating cardiac damage caused by myocardial infarct in a subject in need thereof.

In another aspect, there is provided use of the compound of formula (I) or salt thereof in the manufacture of a medicament for treating cardiac in a subject suffering from chronic heart failure.

In another aspect, there is provided use of the compound of formula (I) or salt thereof in the manufacture of a medicament for restoring or improving cardiac function following myocardial infarct in a subject in need thereof.

In another aspect, there is provided use of the compound of formula (I) or salt thereof in the manufacture of a medicament for restoring or improving cardiac function in a subject suffering from chronic heart failure.

In another aspect, there is provided use of the compound of formula (I) or salt thereof in the manufacture of a medicament for enhancing cardiac repair following myocardial infarct in a subject in need thereof.

In another aspect, there is provided use of the compound of formula (I) or salt thereof in the manufacture of a medicament for enhancing cardiac repair in a subject suffering from chronic heart failure.

In another aspect, there is provided use of the compound of formula (I) or salt thereof in the manufacture of a medicament for treatment of cardiac dysfunction following myocardial infarction in a subject in need thereof.

In another aspect, there is provided use of the compound of formula (I) or salt thereof in the manufacture of a medicament for treatment of cardiac dysfunction in a subject suffering from chronic heart failure.

In another aspect, there is provided use of the compound of formula (I) or salt thereof in the manufacture of a medicament for treatment of persistent angina following myocardial infarction in a subject in need thereof.

In another aspect, there is provided use of the compound of formula (I) or salt thereof in the manufacture of a medicament for treatment of persistent angina in a subject suffering from chronic heart failure.

In another aspect, there is provided use of the compound of formula (I) or salt thereof in the manufacture of a medicament for treatment of persistent angina in a subject in need thereof.

In another aspect, there is provided use of the compound of formula (I) or salt thereof in the manufacture of a medicament for improving survival, decreasing arrhythmias, and/or increasing ventricular contraction and compliance following myocardial infarction in a subject in need thereof.

In another aspect, there is provided use of the compound of formula (I) or salt thereof in the manufacture of a medicament for improving survival, decreasing arrhythmias, and/or increasing ventricular contraction and compliance in a subject suffering from chronic heart failure.

In another aspect, there is provided use of the compound of formula (I) or salt thereof in the manufacture of a medicament for prolonging survival of a subject following myocardial infarction.

In another aspect, there is provided use of the compound of formula (I) or salt thereof in the manufacture of a medicament for prolonging survival of a subject suffering from chronic heart failure.

In another aspect, there is provided use of the compound of formula (I) or salt thereof in the manufacture of a medicament for preventing the development of severe heart failure in a subject following myocardial infarction.

In another aspect, there is provided use of the compound of formula (I) or salt thereof in the manufacture of a medicament for preventing the development of severe heart failure in a subject suffering from chronic heart failure.

In another aspect, there is provided use of the compound of formula (I) or salt thereof in the manufacture of a medicament for treatment of chronic heart failure in a subject in need thereof.

In the methods and uses of the invention described above, the compound of formula (I) or salt thereof may be administered to a subject in need thereof in an amount of between about 0.1 to 100 mg/kg, or an amount of between about 0.1 to 0.5, 0.1 to 1, 0.1 to 2, 0.1 to 3, 0.1 to 4, 0.1 to 5, 0.1 to 6, 0.1 to 7, 0.1 to 8, 0.1 to 10, 0.5 to 1, 0.5 to 2, 0.5 to 3, 0.5 to 4, 0.5 to 5, 0.5 to 6, 0.5 to 7, 0.5 to 8, 0.5 to 9, 0.5 to 10, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 0, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, 9 to 10, 5 to 10, 5 to 20, 5 to 30, 5 to 40, 5 to 50, 5 to 60, 5 to 70, 5 to 80, 5 to 90, 5 to 100, 10 to 20, 10 to 30, 10 to 40, 10 to 50, 10 to 60, 10 to 70, 10 to 80, 10 to 90, 10 to 100, 20 to 30, 20 to 40, 20 to 50, 20 to 60, 20 to 70, 20 to 80, 20 to 90, 20 to 100, 30 to 40, 30 to 50, 30 to 60, 30 to 70, 30 to 80, 30 to 90, 30 to 100, 40 to 50, 40 to 60, 40 to 70, 40 to 80, 40 to 90, 40 to 100, 50 to 60, 50 to 70, 50 to 80, 50 to 90, 50 to 100, 60 to 70, 60 to 80, 60 to 90, 60 to 100, 70 to 80, 70 to 90, 70 to 100, 80 to 90, 80 to 100, or 90 to 100 mg/kg. In the methods and uses of the invention described above, the compound of formula (I) may be administered to a subject in need thereof in an amount of 0.1 mg/kg, or about 0.5, 1, 1.5, 2, 2.5, 3, 3.5, 4, 4.5, 5, 5.5, 6, 6.5, 7, 7.5, 8, 8.5, 9, 9.5, 10, 20, 30, 40, 50, 60, 70, 80, 90, or 100 mg/kg.

In the methods and uses of the invention described above, the compound of formula (I) or salt thereof may be administered to a subject in need thereof at a frequency of twice per day, once per day, twice per week, once per week, once every 2 weeks, monthly, once every two months, or once every 6 months. In the methods and uses of the invention described above, the duration of treatment with a compound of formula (I) or salt thereof may be 1 day, 2 days, 3 days, 4 days, 5 days, 6 days, 7 days, 14 days, one month, two months, three months, four months, five months, six months, seven months, eight months, nine months, ten months, eleven months, or twelve months. Any of the above dosage frequencies may be combined with any of the above durations of treatment. For example, the compound of formula (I) may be administered at a frequency of once per day for a duration of 5 days.

As discussed above, the compounds of the invention unexpectedly provide activity which mimics that of PDGF while avoiding excessive and harmful fibrogenesis.

For example, administration of the compound of formula (I) or salt thereof, or the pharmaceutical composition of the invention to the subject does not significantly change the fibrotic area of the post infarct heart. That is, after administration of the compound of formula (I) or salt thereof, or the pharmaceutical composition of the invention, the fibrotic area of the post infarct heart is not increased by more than 5%, or by more than 10, 15, 20, 30, 40 or 50% compared to the size of cardiac fibrosis post infarct but prior to administration of the compound of formula (I) or salt thereof, or the pharmaceutical composition of the invention. In some embodiments, the fibrotic area of the post infarct heart may be decreased compared to the size of cardiac fibrosis post infarct but without administration of the compound of formula (I) or salt thereof, or the pharmaceutical composition of the invention. The fibrotic area of the post infarct heart may be measured by Gomori Trichrome staining on the infarcted heart.

Furthermore, administration of the compound of formula (I), or the pharmaceutical composition of the invention to the subject does not significantly increase the size of cardiac fibrosis post infarct compared to administration of a corresponding dosage of a homodimer of the platelet derived growth factor (PDGF)—B chain which is not conjugated to human serum albumin (HSA). By ‘corresponding dosage’ it is meant a dosage of the compound of formula (I) determined to have the same potency as PDGF−AB or PDGF−BB in in vitro assay. ‘Corresponding dosage’ may also mean the dosage of the compound of formula (I) required to achieve a plasma concentration which provides a therapeutic effect in vivo. By ‘does not significantly increase’ it is meant administration of the compound of formula (I) or salt thereof, or the pharmaceutical composition of the invention does not increase the size of cardiac fibrosis by more than 5%, or by more than 10, 15, 20, 30, 40 or 50% compared to the size of cardiac fibrosis post infarct but without administration of the compound of formula (I) or salt thereof, or the pharmaceutical composition of the invention. In some embodiments, the size of the cardiac fibrosis may be decreased compared to the size of cardiac fibrosis post infarct but without administration of the compound of formula (I) or salt thereof, or the pharmaceutical composition of the invention.

### EXAMPLES

### Preparation of Compounds

### Reagents and Solvents

Peptide-grade N,N-dimethylformamide (DMF) and dichloromethane (DCM) were purchased from RCI Labscan and Merck, respectively. Acetonitrile (MeCN) for chromatography was purchased as ‘gradient grade’ from Sigma-Aldrich and ultrapure water was from a Merck Millipore Direct-Q 5 water purification system. All solvents for chromatography were supplemented with formic acid (FA) purchased from Sigma-Aldrich. All standard Fmoc-protected amino acids were purchased from Mimotopes. Rink amide resins for peptide synthesis were purchased from Mimotopes. Automated SPPS was carried out on Biotage Syrol.

General solid phase peptide synthesis procedure:

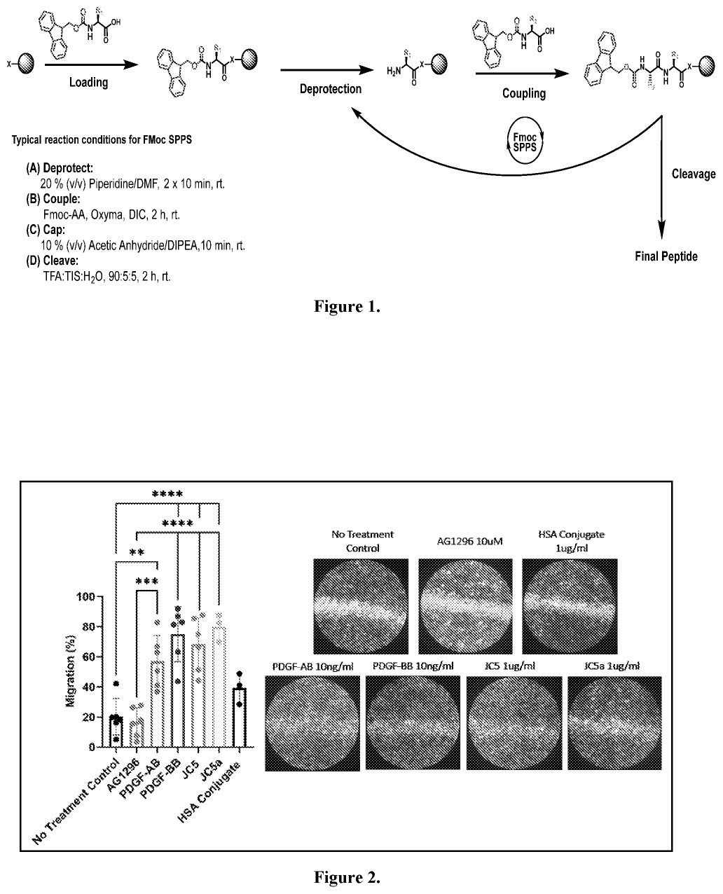

The resin (164 mg, 100 μmol, 0.61 mmol g−1, 1 eq.) was treated with 40 vol. % piperidine (1.6 mL) in DMF for 3 min, drained, and then treated with 20 vol. % piperidine in DMF for 10 min (1.6 mL), drained, and washed with DMF (4×1.6 mL). The resin was then treated with a solution of Fmoc-Xaa-OH (400 μmol, 4 eq.) and Oxyma (57 mg, 400 μmol, 4 eq.) in DMF (800 μL), followed by a solution of DIC (63 μL, 400 μmol, 4 eq.) in DMF (800 μL) and shaken at rt for 1 h. The resin was then drained and washed with DMF (4×1.6 mL) before being treated with a solution of 5 vol. % Ac₂O and 10 vol. % iPr₂NEt in DMF (1.6 mL) for 5 min at rt, drained, washed with DMF (4×1.6 mL) and drained. A general scheme is shown in FIG. 1.

Peptide cleavage from resin: Resin-bound peptide was shaken in a cleavage solution of TFA/TIS/H₂O (90:5:5 v/v/v) for 2 h at rt. The crude product was drained, and the resin rinsed with cleavage cocktail (˜2 mL). These solutions were combined and concentrated to <1 mL under nitrogen flow. To precipitate the free peptide, diethyl ether (14 mL) was added to the crude concentrate. The resultant suspension was centrifuged for 4 min at 7000 rcf to pellet the free peptide. The supernatant was then decanted, and the precipitation process repeated once more. The crude peptide was dried under nitrogen flow, then re-dissolved in 50% v/v aq. MeCN (˜6 mL) for cyclisation.

### Disulfide Formation

Crude or purified peptide fragments were dissolved in saturated aqueous ammonium carbonate solution and stirred under air for 24 hours. Upon completion as determined by UHPLC the solution was concentrated under a flow of nitrogen. The residue was then dissolved in DMF for HPLC purification.

### Preparative Chromatography

Reversed-phase high performance liquid chromatography (HPLC) was performed on a Waters 600E multi-solvent delivery system fitted with a Rheodyne 7725i injection valve (5 mL loading loop), a Waters 500 pump and a Waters 490E programmable wavelength detector operating at 214 nm and 230 nm. Preparative reversed-phase HPLC was performed using a Waters semi-preparative Sunfire OED C18 column (5 m, 19×150 mm) at a flow rate of 15 mL min⁻¹. All preparative HPLC used a mobile phase of ultrapure (type 1) water (Solvent A) and MeCN (Solvent B) supplemented with 0.1 vol % formic acid or trifluoroacetic acid (TFA) on gradients as specified.

### Analytical Chromatography

Liquid Chromatography-Mass Spectrometry (UPLC) was performed on a Shimadzu 2020 UPLC instrument with a Nexera X2 LC-30AD pump, Nexera X2 SPD-M30A UV/Vis diode array detector and a Shimadzu 2020 (ESI) mass spectrometer operating in either positive or negative mode. Separations were performed on a Waters Acquity BEH300 1.7 μm, 2.1×50 mm (C18) column at a flow rate of 0.6 mL min⁻¹. All separations were performed using a mobile phase of 0.1 vol % formic acid in water (Solvent A) and 0.1 vol % formic acid in MeCN (Solvent B) using gradients as specified. Analytical reversed-phase HPLC was performed on a Waters Acquity UPLC system equipped with a PDA λ detector (λ=210-400 nm). Separations were performed on a Waters Acquity BEH300 1.7 μm, 2.1×50 mm (C18) column at a flow rate of 0.6 mL min⁻¹. All separations were performed using a mobile phase of 0.1 vol % TFA in water (Solvent A) and 0.1 vol % TFA in MeCN (Solvent B) using gradients as specified.

### Matrix-Assisted Laser Desorption Ionisation (MALDI) Mass Spectrometry

Matrix-assisted laser desorption/ionisation time-of-flight (MALDI-TOF) mass spectra were recorded on a Bruker Autoflex TM Speed MALDI-TOF instrument operating in linear, positive mode using a matrix of 10 mg/mL sinapinic acid in H₂O:MeCN (1:1 v/v) with no TFA.

### Compounds

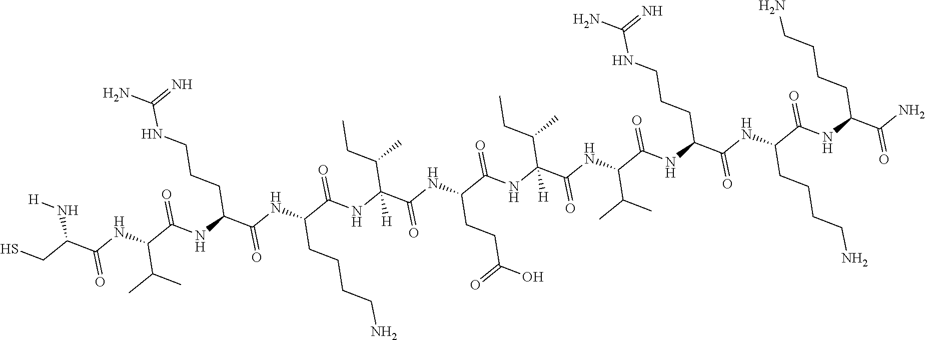 [SMILES: CC[C@H](C)[C@H](NC(=O)[C@H](CCCCN)NC(=O)[C@H](CCCNC(=N)N)NC(=O)[C@@H](NC(=O)[C@@H](N)CS)C(C)C)C(=O)N[C@@H](CCC(=O)O)C(=O)N[C@H](C(=O)N[C@H](C(=O)N[C@@H](CCCNC(=N)N)C(=O)N[C@@H](CCCCN)C(=O)N[C@@H](CCCCN)C(N)=O)C(C)C)[C@@H](C)CC]

PDGF Loop III monomer was synthesised via Fmoc-strategy SPPS as specified in general methods. The linear sequence N′-CVRKIEIVRKK-C′ was generated by automated SPPS on Rink amide resin (204 mg, 100 μmol, capacity: 0.49 mmolg⁻¹). A 50 μmol portion was cleaved from resin as described in the general methods. The crude linear peptide was purified by semi-preparative RP-HPLC (0 to 30% B+0.1% formic acid over 30 min). The appropriate fractions were combined and lyophilised to afford PDGF Loop III monomer as a white solid (22.6 mg, 32%). HPLC Rₜ=16.64 mins (1 to 40% B+0.1% TFA over 30 mins). LRMS (ESI+): m/z=1371.5 [M+H]⁺.

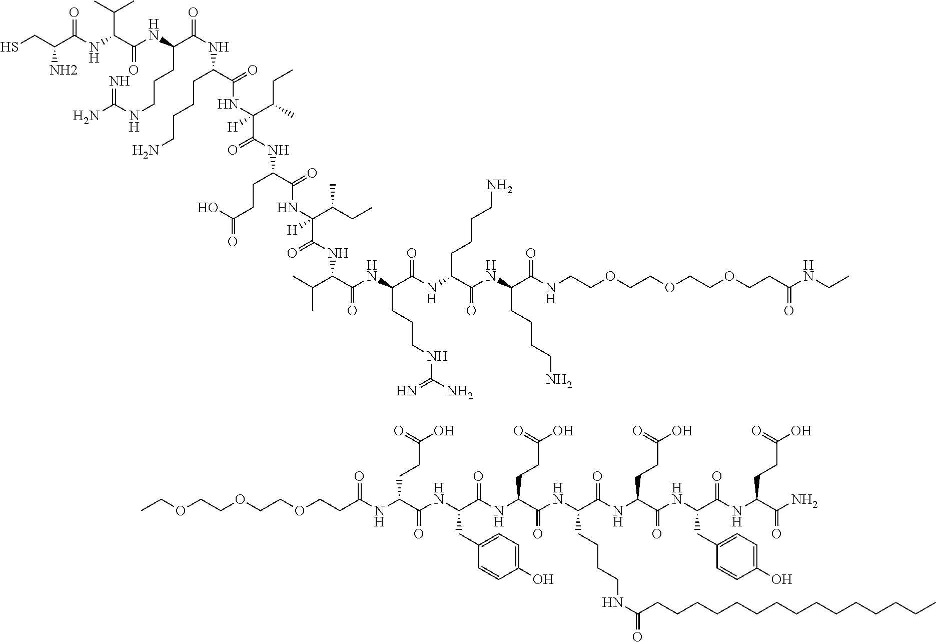

PDGF Loop III HSA monomer was synthesised via Fmoc-strategy SPPS as specified in general methods. The linear sequence N′-CVRKIEIVRKK(Peg3)₂EYEK(Palm)EYE-C′ was generated by automated SPPS on Rink amide resin (204 mg, 100 μmol, capacity: 0.49 mmolg⁻¹). A 50 μmol portion was cleaved from resin as described in the general methods. The crude linear peptide was used without further purification. PDGF Loop III HSA monomer as a white solid (53 mg, 36%). HPLC Rₜ=34.61 mins (1 to 50% B+0.1% TFA over 40 mins). LRMS (ESI+): m/z=1494.2 [M+2H]²⁺.

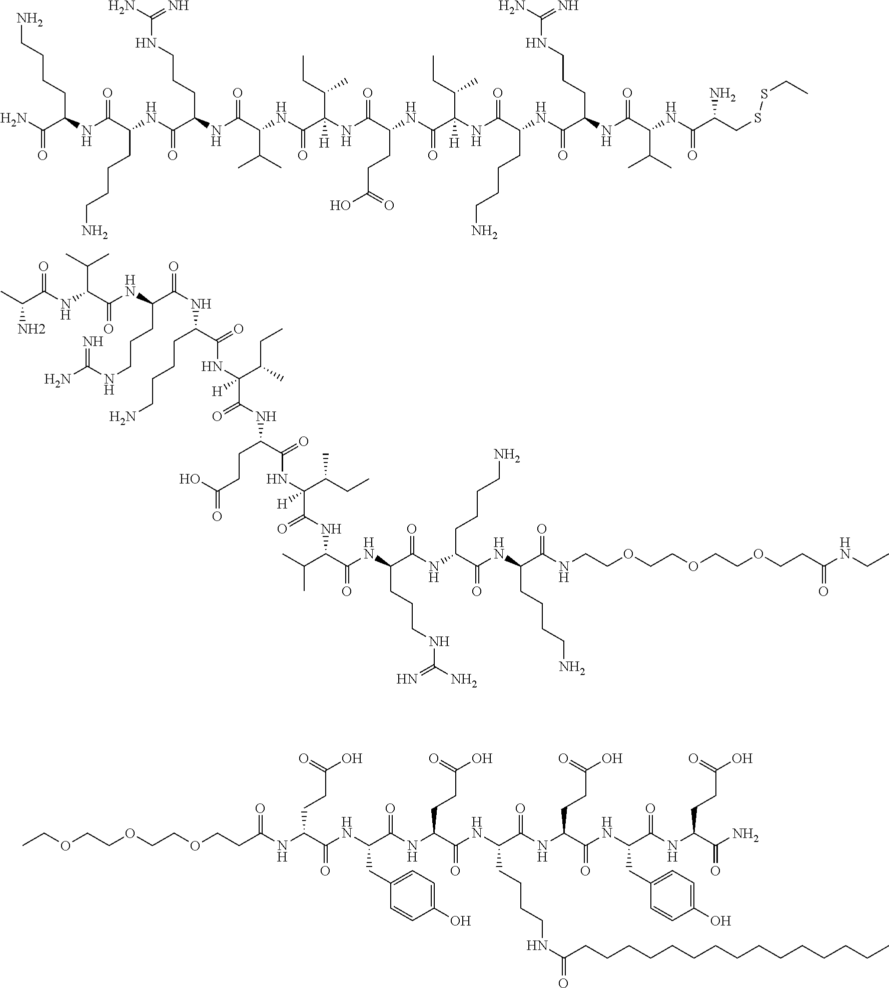

PDGF Loop III dimer HSA (CVRKIEIVRKK)₂(Peg3)₂EYEK(Palm)EYE-C′ was synthesised via disulfide bond formation as specified in general methods. The linear sequence N′-CVRKIEIVRKK-C′ (12.2 mg (0.009 mmol) and N′—(CVRKIEIVRKK)₂(Peg3)₂EYEK(Palm)EYE-C′ (27 mgs (0.009 mmol) was dissolved in DMF (1 mL) and to the solution was added saturated aqueous NH₄⁺CO₃⁻ and the reaction mixture was stirred for 48 hours. The crude linear peptide was purified by semi-preparative RP-HPLC (0 to 30% B+0.1% formic acid over 30 min). The appropriate fractions were combined and lyophilised to afford PDGF Loop III dimer HSA as a white solid (3.2 mg, 8%). HPLC Rₜ=21.77 mins (1 to 80% B+0.1% TFA over 40 mins). LRMS (ESI+): m/z=1452.7[M+3H]³⁺.

MALDI-TOF (ESI+): m/z=4357.1 [M+H]⁺.

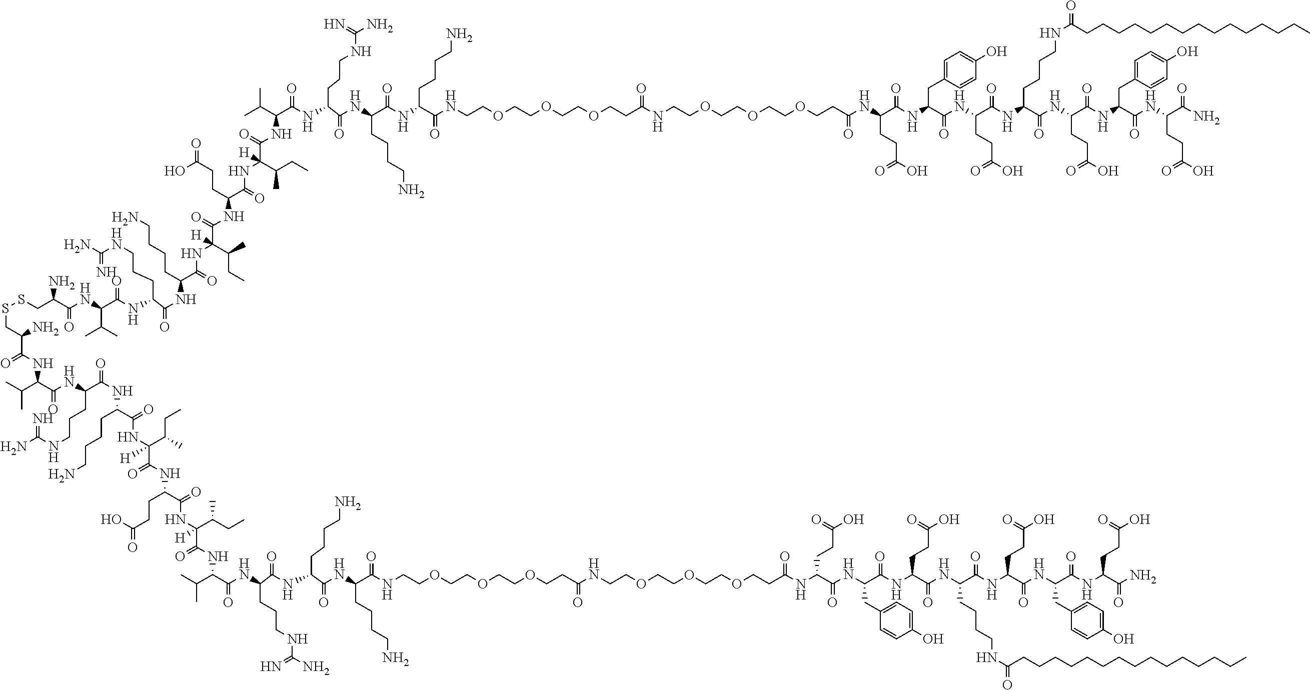

PDGF Loop III dimer USA dimer (CVRKIEIVRKK)₂(Peg3)₂(EYEK(Palm)EYE)₂-C′ was synthesised via disulfide bond formation as specified in general methods. The linear sequence (CVRKIEIVRKK)₂(Peg3)₂EYEK(Palm)EYE-C′ (10 mg, 0.0032 mmol) was dissolved in DMF (1 mL) and to the solution was added saturated aqueous NH₄+CO₃⁻ (1 mL) and the reaction mixture was stirred for 48 hours. The crude linear peptide was purified by semi-preparative RP-HPLC (0 to 30% B+0.1% formic acid over 30 min). The appropriate fractions were combined and lyophilised to afford PDGF Loop III dimer HSA Dimer as a white solid (3.6 mg, 36%). HPLC Rₜ=25.33 mins (1 to 80% B+0.1% TFA over 40 mins). LRMS (ESI+): m/z=1493.7 [M+4H]⁴⁺. MALDI-TOF (ESI+): m/z=5973.2 [M+H]⁺.

### In Vitro Studies

The function of Compounds Ia (also referred to herein as JC5) and Ib (also referred to herein as JC5a) has been tested in vitro on the continuous cell line, C2C12 mouse myoblasts. In these in vitro assays recombinant PDGF−AB and −BB have been used as positive controls along with a serum starved no treatment control and the PDGF receptor inhibitor AG1296 co-treated with PDGF−AB as a negative control. The in vitro assays were designed to assess the major known functions of PDGF whilst also considering functions that are relevant to its in vivo applications. These functions were chemotaxis, collagen contraction, vascular tube formation and activation of the PDGF receptors via phosphorylation of Akt and Erk.

Chemotaxis was assessed by making a scratch through a 2D culture of C2C12 cells following treatment with the four controls and the peptides JC5 and JC5a. They were then incubated for 24 hours at 37° C. Migration of the cells across the scratch at the conclusion of the incubation was measured and compared to the relevant controls described above. PDGF is a well-established mediator of chemotaxis and is expected to increase migration of the cells across the scratch over the course of 24 hours post treatment.

A significant increase in migration of C2C12 cells treated with recombinant PDGF-BB and peptides JC5 and JC5a is shown in FIG. 2. The migration of the positive control PDGF−BB treated cells was 75.02% (±18.14), not significantly higher than migration of JC5 treated cells at 68.28% (±17.80) and was lower than JC5a at 79.88% (±9.096). The no treatment control group migrated 20.32% (±12.08) similarly to the negative control AG1296+PDGF−AB treated cells that migrated 16.57% (±9.602). PDGF−BB treated cells had a 54.70% (Mean Diff.) increase when compared to the no treatment control (p<0.0001) and a 58.45% (Mean Diff) increase when compared to the negative control (p<0.0001). Cells treated with novel mimetic peptide JC5 had a 47.96% (Mean Diff.) increase when compared to the no treatment control group (p<0.0001) and a 51.71% (Mean Diff) increase when compared to the negative control (p<0.0001). Cells treated with novel mimetic peptide JC5a had a 59.56% (Mean Diff.) increase when compared to the no treatment control group (p<0.0001) and a 62.71% (Mean Diff) increase when compared to the negative control (p<0.0001). In summary, our novel peptide demonstrated a chemotactic function similar to recombinant human PDGF−AB and PDGF-BB.

Collagen contraction is an important function during wound healing of many tissues including the heart. This can be simulated in vitro using a collagen gel contraction assay. This was assessed using C2C12 cells treated with the positive controls 10 ng/ml recombinant PDGF-BB and PDGF−AB, negative control 10 μM AG1296 co-treated with 10 ng/ml recombinant PDGF−AB, a serum starved no treatment control and 1 μg/ml of the peptides JC5 and JC5a. C2C12 mouse myoblasts were seeded in a 3D collagen gel crosslinked with NaOH and incubated at 37° C. for 24 hours following treatment with the four controls and peptides JC5 and JC5a.

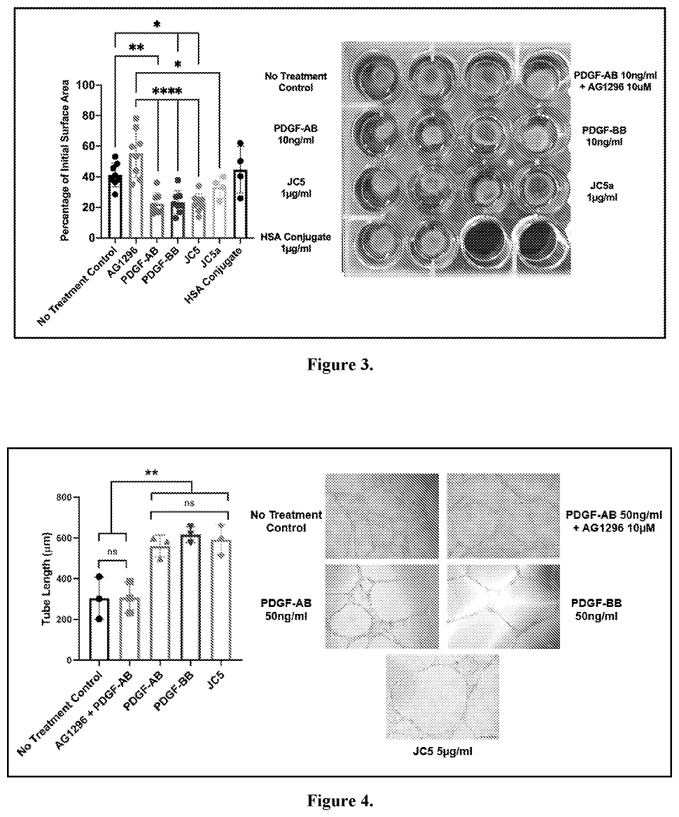

Increased collagen contraction is known to occur following treatment with PDGF and was observed in this assay following treatment with PDGF-BB, PDGF−AB, and peptides JC5 and JC5a (FIG. 3). The increase in collagen contraction was statistically significant when compared to our negative control AG1296+PDGF−AB whilst, PDGF−AB, PDGF−BB and JC5 were statistically significant when compared to the no treatment control. These results are consistent with other reported data on PDGF in collagen contraction assays. Following a 24-hour incubation C2C12 cells treated with PDGF−AB had contracted the collagen gel to 22.46% (±7.081) of its initial surface area, whilst PDGF−BB had contracted to 23.42% (±7.522), JC5 had contracted to 23.01% (±6.040) and JC5a had contracted to 33.28% (±6.979). The negative control AG1296 co-treated with PDGF−AB contracted to 55.25% (±15.61) which was 32.79% (Mean Diff.) less than PDGF−AB (p>0.0001), 31.83% (Mean Diff) less than PDGF-BB (p>0.0001), 32.23% (Mean Diff) less than our novel mimetic peptide JC5 (p>0.0001), and 21.96% (Mean Diff) less than our novel mimetic peptide JC5a (p=0.0120). The no treatment control contracted to 41.27% (±7.675) which was 18.82% (Mean Diff) less than PDGF−AB (p=0.0073), 17.86% (Mean Diff.) less than PDGF−BB (p=0.0126), and 18.26% (Mean Diff.) less than our novel mimetic peptide JC5 (p=0.0100). In summary, our novel mimetic peptides have similar collagen contracting ability to recombinant human PDGF−AB and PDGF-BB.

PDGF has a well-established role in angiogenesis in vivo. To simulate this function in vitro a vascular tube forming assay on primary human coronary artery endothelial cells (HCAECs) was carried out. Cells were seeded on top of 3D growth factor reduced Geltrex basement membrane extract and incubated for 24 hours following treatment with the PDGF−AB, PDGF-BB, no treatment control, AG1296+PDGF−AB negative control, and peptides JC5 and JC5a. Over the course of 24 hours the cells form tubes throughout the gel. PDGF is reported to facilitate tube formation of endothelial cells in in vitro 3D assays.

Cells treated with 50 ng/ml of PDGF−AB, PDGF-BB, and 5 μg/ml of JC5 produced longer more uniform tubes than untreated cells and cells treated with the negative control 10 μM PDGF receptor inhibitor AG1296 co-treated with 50 ng/ml PDGF−AB (FIG. 4). The positive controls PDGF−AB and PDGF−BB produced tubes that were 560.7 μm (±53.93) and 616.3 μm (±39.07), respectively. These were not significantly different to JC5 that produced tubes that were 591.3 μm (±73.23). The no treatment control and AG1296+PDGF−AB negative control produced tubes that were 305 μm and 307.7 μm, respectively. The PDGF−AB treatment group had an increased tube length of 255.7 μm (Mean Diff.) compared to the no treatment control (p=0.0062) and 253 μm (Mean Diff.) compared to the negative control (p=0.0068). The PDGF-BB treatment group had an increased tube length of 311.3 μm (Mean Diff.) compared to the no treatment control (p=0.0013) and 308.7 μm (Mean Diff.) compared to the negative control (p=0.0014). The JC5 treatment group had an increased tube length of 286.3 μm (Mean Diff.) compared to the no treatment control (p=0.0025) and 283.7 μm (Mean Diff) compared to the negative control (p=0.0027). In summary, JC5 has similar vascular tube forming ability to recombinant human PDGF−AB and PDGF-BB.

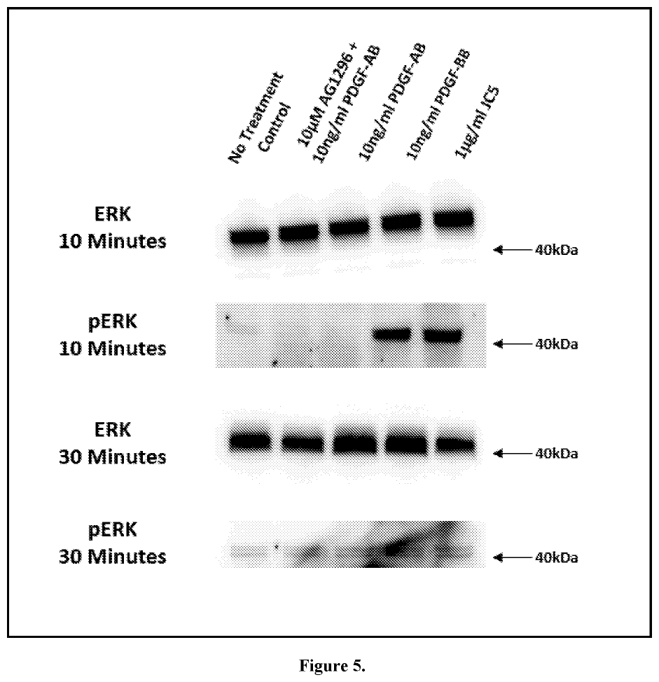

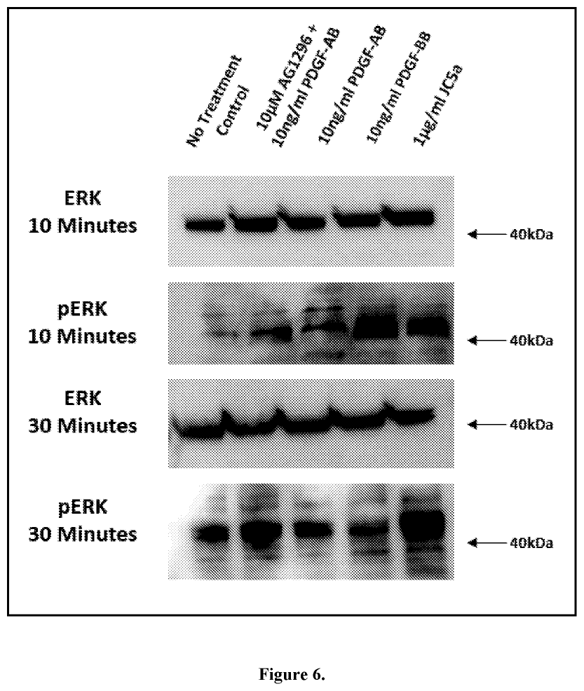

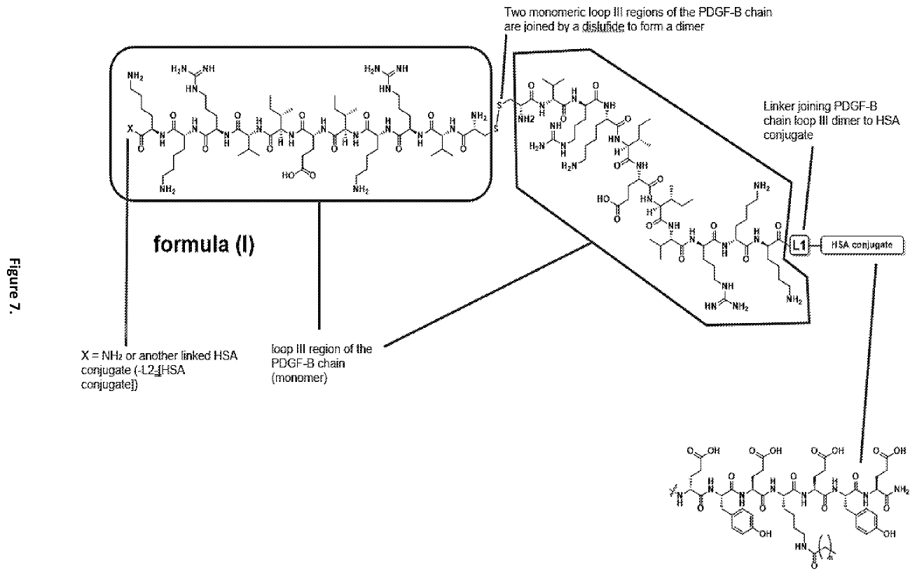

PDGF receptor activation takes place via autophosphorylation of tyrosine and threonine residues at key sites throughout the intracellular domain. Autophosphorylation engages different signaling pathways, including ERK (MAPK) which participates in proliferation, cell survival, angiogenesis, and chemotactic activity of cells. Phosphorylation of ERK indicates successful activation of the PDGF receptor. In FIG. 5 phosphorylation of ERK (pERK) can be seen in the PDGF−BB treated group and the JC5 treated group when compared to the no treatment control, AG1296+PDGF−AB negative control and PDGF−AB groups. In FIG. 6, phosphorylation of ERK (pERK) can be seen in the PDGF−BB treated C2C12 cells and the JC5a treated cells when compared to the no treatment control, AG1296+PDGF−AB negative control and PDGF−AB cells. We have also observed a preserved phosphorylation of ERK (pERK) following treatment with our PDGF mimetic peptide JC5a for 30 minutes. These results indicate successful activation of the PDGF receptor following treatment with JC5 and JC5a. Further it is noted that the AB ligand requires presence of PDGFRα, which is very lowly expressed in C2C12 cells (Contreras et al), whereas PDGFRβ is expressed at high levels; thus, pERK is induced by PDGF−BB but not PDGF−AB.

The mitotic effects of PDGF mimetic peptides JC5 and JC5a were tested using cultured C3H10T½ mouse embryonic fibroblasts. Proliferation was assayed after a pulse of nucleotide analogue 5-ethynyl-2′-deoxyuridine (EdU) and fluorescent detection using flow cytometry. Cells were treated with 20 ng/ml PDGF−BB or PDGF−AB, and 2 μg/ml of peptides JC5 or JC5a, in the presence of 1% fetal bovine serum (FBS) and DMEM for 24 hr. 10% FBS was used as a positive control, and co-treatments with specific PDGF receptor antagonist, AG1296, was used to ascertain whether proliferation occurred via the PDGF ligand/receptor pathway. Assays were performed in biological triplicate.

Both 10% FBS (positive control) and PDGF−BB stimulated EdU uptake >2 fold (p<0.001) with the latter significantly inhibited by AG1296, confirming stimulation via the PDGF ligand/receptor pathway. However, PDGF−AB, JC5 and JC5a did not significantly stimulate proliferation relative to 1% FBS. Thus, even though the PDGF peptide moiety of JC5 and JC5a were based on the loop III sequence of PDGF-BB, they did not show significant proliferative activity in this assay. The inhibition of EdU levels shown with PDGF−AB and JC5 after cotreatments with AG1296 (p<0.01 and p<0.05, respectively), suggests a weak stimulation of the PDGF ligand/receptor pathway with PDGF−AB and JC5 in the presence of 1% FBS, although at much lower levels than that seen with PDGF-BB. In summary, novel mimetic peptides JC5 and JC5a display an effect on proliferation in C3H₁₀T½ that is significantly less than PDGF-BB. This contrasts the results seen in cell migration, collagen gel contraction and angiogenesis assays, where PDGF−AB, PDGF-BB, JC5 and JC5a show comparable activities (FIGS. 2-4). These data suggest novel biological activities for JC5 and JC5a compared to native recombinant PDGF-BB.

### In Vivo Studies

The in vivo efficacy of the peptides was assessed using a murine model of acute myocardial infarction (MI) established using a surgically induced MI via permanent occlusion of the left anterior descending (LAD) artery along with non-infarcted sham control mice. Cardiac function was monitored using echocardiography with a post MI baseline at day 2 and a day 28 end point followed by tissue collection and calculation of heart weight divided by tibia length. Mice were randomised into treatment groups receiving either 60 μg/kg of the positive control recombinant human PDGF−AB, 32.50 mg/kg of novel mimetic peptide JC5, 32.50 mg/kg of novel mimetic peptide JC5a, or phosphate buffered saline (PBS) as a vehicle control, via implanted minipump, at the time of infarction. In addition to this sham controls were included using non-infarcted healthy mice receiving either 32.50 mg/kg of novel mimetic peptide JC5 or 32.50 mg/kg of novel mimetic peptide JC5a.

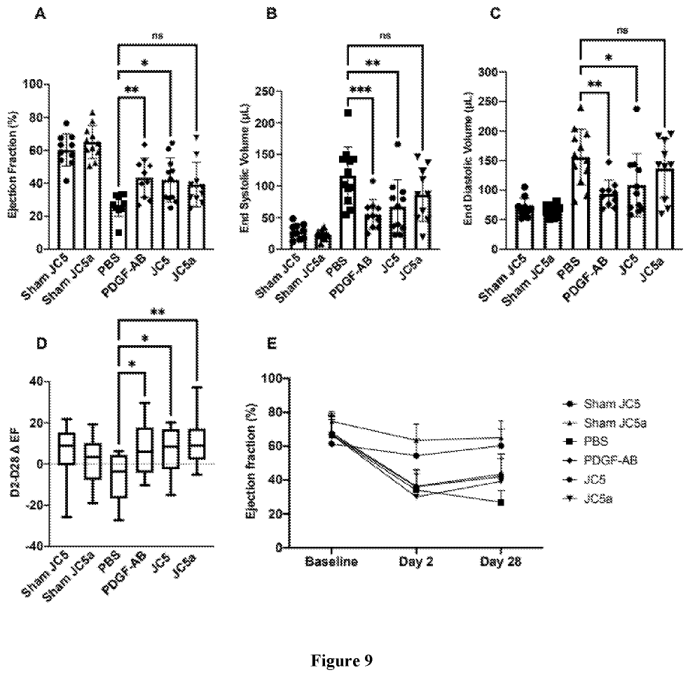

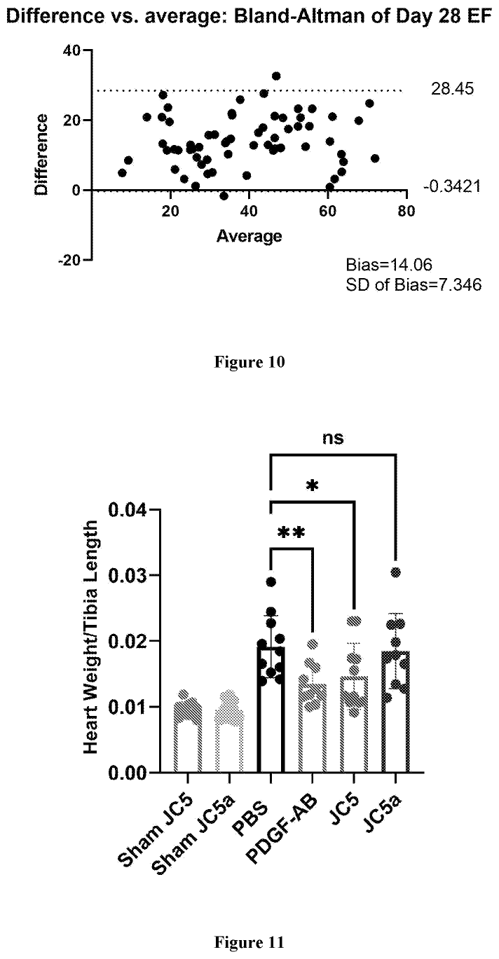

At 28 days post MI mice treated with PDGF−AB (MI) and novel mimetic peptide JC5 (MI) had a significantly increased ejection fraction when compared to mice that received the PBS (MI) vehicle control (FIG. 9A). Mice treated with PDGF−AB (MI) had a day 28 ejection fraction of 43.44% (±11.90) similarly to mice treated with JC5 (MI) that had a day 28 ejection fraction of 42.06% (±13.41) compared to mice that received PBS (MI) that had a day 28 ejection fraction of 26.89% (±6.966) (PDGF−AB p=0.0073, JC5 p=0.0129). Novel mimetic peptide JC5a (MI) demonstrated a trend towards increased ejection fraction but was not significant when compared to PBS (MI) at day 28 (p=0.0655), with an ejection fraction of 39.24% (±13.51). The validity of the day 28 ejection fraction results was confirmed using a Bland-Altman inter-observer variability analysis (FIG. 10). The bias of this analysis was 14.06 (±7.346) and is consistent across all replicates when comparing observer 1 to observer 2. It falls within reported ranges for murine echocardiography of injured hearts (Grune, Blumrich et al. 2018, Hume, Kanagalingam et al. 2023). Both observers were blinded for the duration of analysis.

Left ventricular ejection fraction (LVEF) is directly proportionate to the difference between left ventricle end diastolic volume and the left ventricular end systolic volume (LVEDV-LVESV) and inversely proportionate to left ventricular end diastolic volume (LVEDV). The value is created by the following equation LVEF=(LVEDV-LVESV)/LVEDV. Increased end diastolic volume (EDV) indicates possible dilation of the ventricle whilst increased end systolic volume (ESV) indicates a possible decrease in contractile function. In mice treated with positive control PDGF−AB (MI) and novel mimetic peptide JC5 (MI) there was a significant decrease in both ESV and EDV at day 28 when compared to the vehicle control, PBS (MI), cohort. The decreased ESV of the PDGF−AB (MI) cohort at day 28 aligned with what was expected of the positive control with a mean ESV of 55.04 μL (±23.66), 61.54 μL (mean diff.) less than the PBS (MI) cohort (p=0.0003). The PDGF−AB (MI) cohort had a mean EDV of 93.75 μL (±23.17) whilst the JC5 (MI) cohort had a mean EDV of 108.6 μL (±53.37). These were both a significant decrease from the PBS (MI) cohort with a mean EDV of 155.9 μL (±47.15) (JC5 p=0.0185, PDGF−AB p=0.0016). JC5 (MI) mouse cohort had a mean ESV of 67.01 μL (±42.94) which was 49.58 μL (mean difference) less than the PBS (MI) cohort with a mean ESV of 116.6 μL (±45.00) (p=0.0032).

Whilst JC5a (MI) did not have a significantly increased ejection fraction at day 28 when compared to PBS (MI), mice treated with JC5a (MI) resulted in the greatest increase from its day 2 ejection fraction. Following treatment with 32.50 mg/kg of novel mimetic peptide JC5a (MI), mice had an average change in ejection of 10.82% from day 2 to day 28. This was 18.02% (mean diff.) greater than that of mice that received the vehicle control, PBS (MI) (p=0.0082). JC5a (MI) mice sustained the greatest decline in ejection fraction from the baseline to day 2 (FIG. 9E) potentially contributing to its lower ejection fraction at day 28 when compared to PDGF−AB (MI) and JC5 (MI). Mice treated with the positive control PDGF−AB (MI) and JC5 (MI) also had a significant increase in ejection fraction from day 2 to day 28 when compared to PBS (MI). Mice treated with PDGF−AB (MI) had an average day 2 to day 28 Δ of 7.104% (±13.19) (PDGF−AB p=0.0393) whilst mice treated with JC5 (MI) had an average day 2 to day 28 Δ of 6.686% (±12.22) (JC5 p=0.0409).

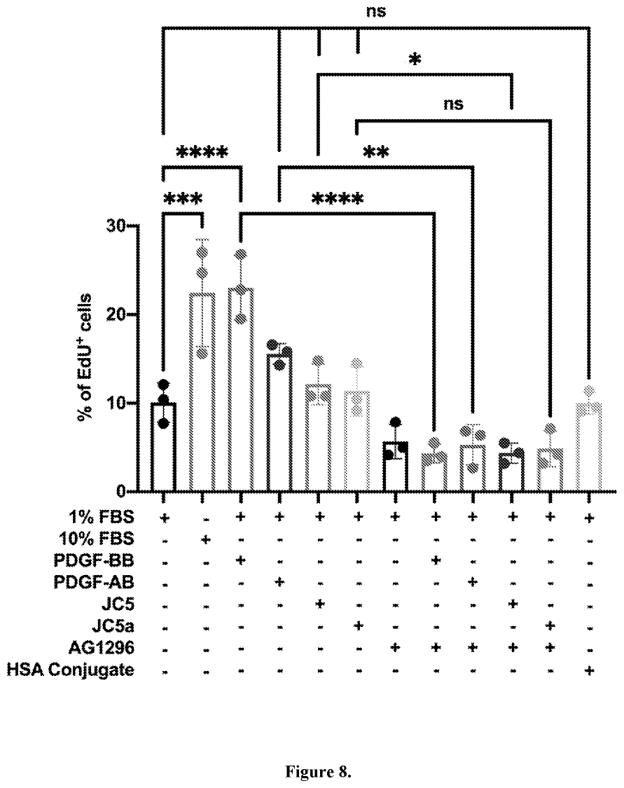

These results indicate a significant improvement in cardiac function of mice treated with the novel mimetic peptides JC5 and JC5a with both peptides performing similarly to the positive control human recombinant PDGF−AB. The significant decrease in ESV and EDV of PDGF−AB and JC5 treated mice indicates that these treatments have an effect on left ventriclular dilation (EDV) and contractility (ESV) leading to increased ejection fraction and improved cardiac function. Whilst both significant, the greater significance seen in ESV suggests that these peptide effects are likely driven more so by effects on left ventriclular contractility than dilation. From this we can conclude that the comparable effects of the mimetic peptides in vitro (FIGS. 2-6 and 8) translate to a maintained biological function of PDGF−AB in vivo (FIGS. 9-11) aligning with previous PDGF−AB studies carried out by our groups (Asli, Xaymardan et al. 2019, Sujitha Thavapalachandran 2020, Hume, Deshmukh et al. 2023).

Supporting the improved cardiac function identified in FIG. 9, mice treated with both the PDGF−AB (MI) positive control and novel mimetic peptide JC5 (MI) resulted in a decreased heart weight when normalised by tibia length when compared to mice that received the vehicle control PBS (MI). JC5 (MI) mice had a mean heart weight/tibia length of 0.01462 (±0.005002) similarly to mice treated with the positive control PDGF−AB (MI) that had a mean heart weight/tibia length of 0.01344 (±0.003075). These were both statistically significant when compared to PBS (MI) mice that had a mean heart weight/tibia length of 0.01913 (±0.004728) (JC5 p=0.0366, PDGF−AB p=0.0066). This indicates that there is a reduced hypertrophy and inflammation in mice treated with novel mimetic peptide JC5 (MI) and positive control PDGF−AB (MI) when compared to PBS vehicle control (MI) mice.

### REFERENCES

Asli, N. S., et al. (2019). “PDGFRa signaling in cardiac fibroblasts modulates quiescence, metabolism and self-renewal, and promotes anatomical and functional repair.” BioRxiv.
Contreras et al, Cellular Signalling 202184:110036.
Grune, J., et al. (2018). “Evaluation of a commercial multi-dimensional echocardiography technique for ventricular volumetry in small animals.” Cardiovasc Ultrasound 16(1): 10.
Hume, R. D., et al. (2023). “PDGF−AB Reduces Myofibroblast Differentiation Without Increasing Proliferation After Myocardial Infarction.” JACC: Basic to Translational Science.
Hume, R. D., et al. (2023). “Tropoelastin Improves Post-Infarct Cardiac Function.” Circ Res 132(1): 72-86.
Lin et al (2007) Growth Factors 25(2): 87-93).
Sujitha Thavapalachandran, S. M. G., Robert D. Hume, Thi Yen Loan Le, Kalyan Raguram, James E. Hudson, Jim Pouliopoulos, Gemma A. Figtree, Rafael P. Dye, Anthony M. Barry, Paula Brown, Juntang Lu, Sean Coffey, Scott H. Kesteven, Richard J. Mills, Fairooj N. Rashid, Elena Taran, Pramesh Kovoor, Liza Thomas, Alan Robert Denniss, Eddy Kizana, Naisana S. Asli, Munira Xaymardan, Michael P. Feneley, Robert M. Graham, Richard P. Harvey, James J. H. Chong (2020). “Platelet-derived growth factor−AB improves scar mechanics and vascularity after myocardial infarction.” Science Translational Medicine 12.

1. A compound of formula (I) or a salt thereof: 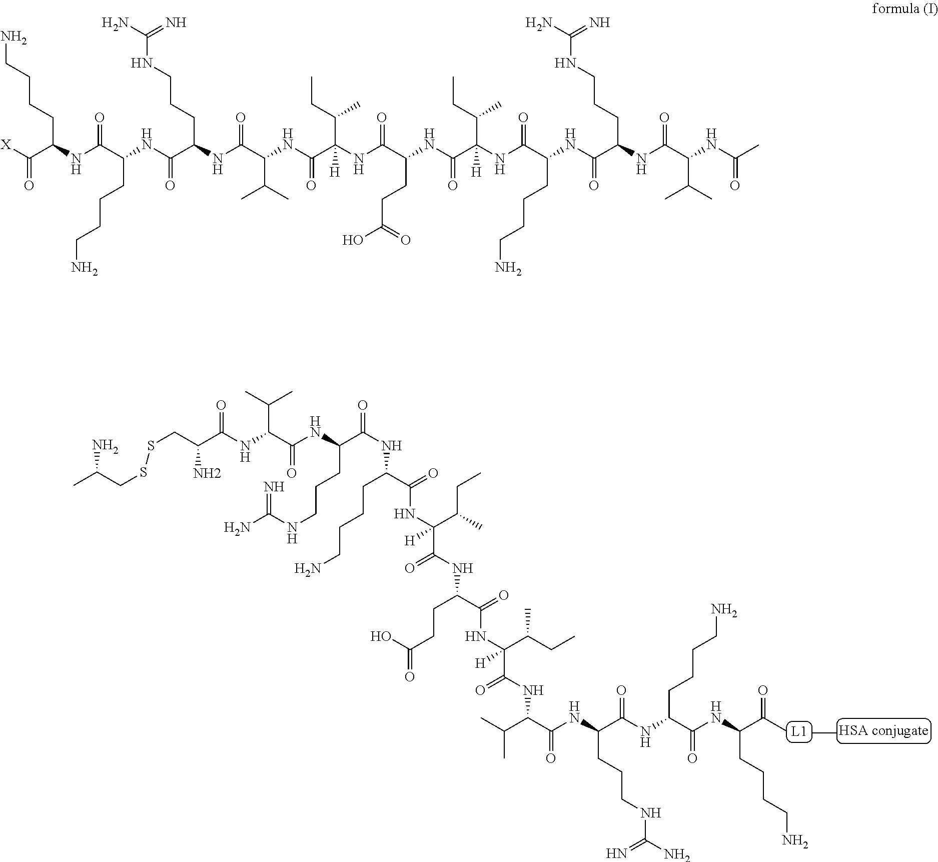
wherein X═NH₂ or -L2-[HSA conjugate], wherein 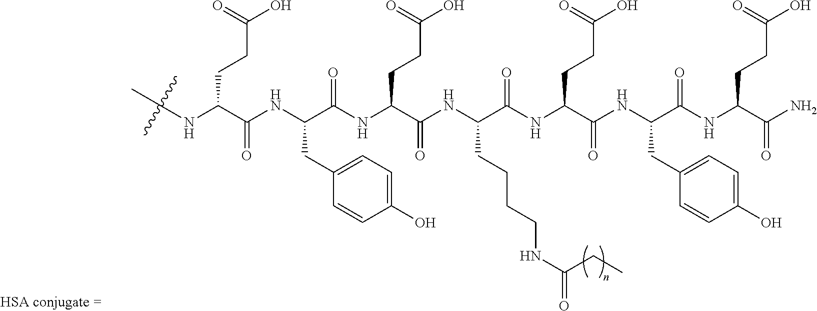
wherein L1 and L2 are independently selected from the group consisting of —HN(C₂-C₆₀ alkyl)C(O)—, —HN(C₂-C₆₀ alkaryl)C(O)—, —HN(C₂-C₆₀ alkenyl)C(O)—,
—HN(CH₂)ᵣ(CH₂CH₂O)ₒ(CH₂)ᵣC(O)—, —HN(CH₂)ᵣ(O(CH₂)ₘC(O))ₒ(CH₂)ᵣC(O)—, —HN(CH₂)ᵣ ((NH(CH₂)ₘC(O))ₒ)(CH₂)ᵣC(O)—, and 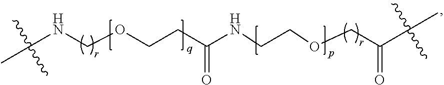
wherein n=1 to 20, o=1 to 60, r=2 to 20, m=1 to 60, p=1 to 60, and q=1 to 60.

2. The compound of claim 1, wherein L1 and L2 are independently selected from the group consisting of —HN(C₂-C₂₀ alkyl)C(O)—, —HN(C₂-C₂₀ alkaryl)C(O)—, —HN(C₂-C₂₀ alkenyl)C(O)—,
—HN(CH₂)ᵣ(CH₂CH₂O)ₒ(CH₂)ᵣC(O)—, —HN(CH₂)ᵣ(O(CH₂)ₘC(O))ₒ(CH₂)ᵣC(O)—, —HN(CH₂)ᵣ ((NH(CH₂)ₘC(O))ₒ)(CH₂)ᵣC(O)—, and 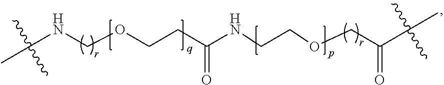
wherein n=1 to 20, o=1 to 20, r=2 to 20, m=1 to 20, p=1 to 20, and q=1 to 20.

3. The compound of claim 1, wherein L1 and L2 are

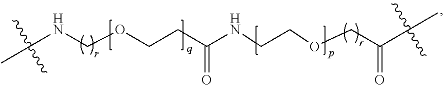

4. The compound of any one of claims 1 to 3, wherein n=15.

5. The compound of any one of claims 1 to 4, wherein r=2.

6. The compound of any one of claims 1 to 5, wherein L1 and L2 are

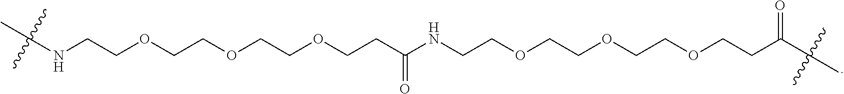 [SMILES: *NCCOCCOCCOCCC(=O)NCCOCCOCCOCCC(*)=O]

7. The compound of any one of claims 1 to 6, selected from the group consisting of:

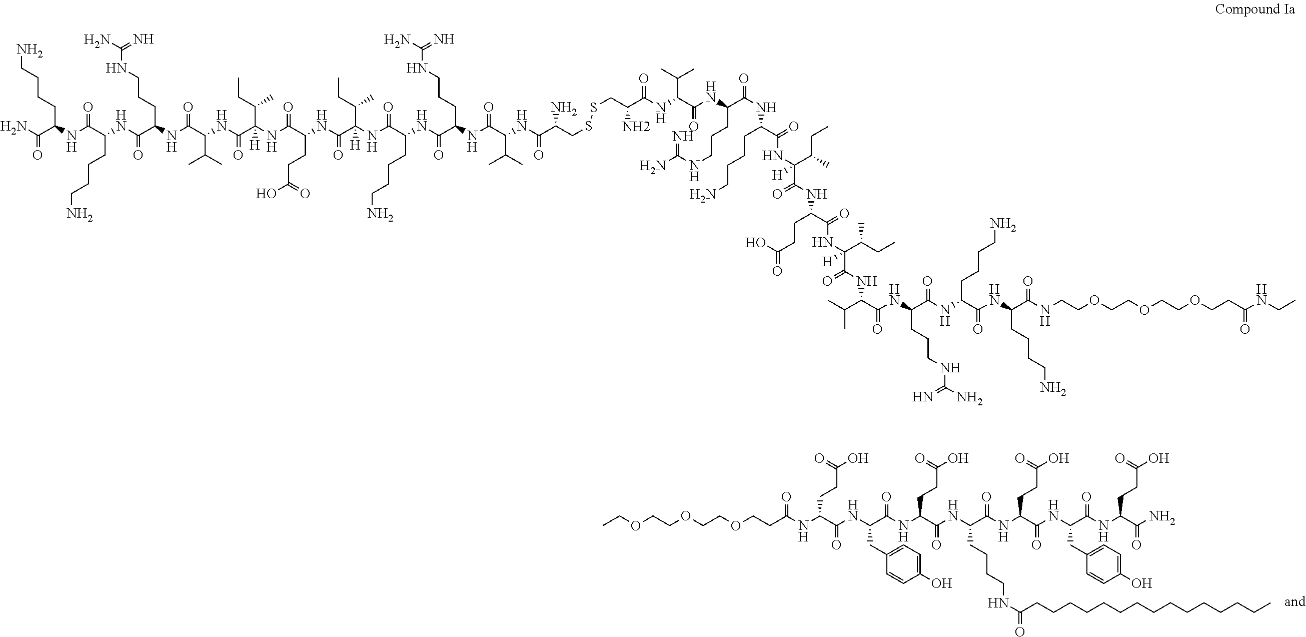

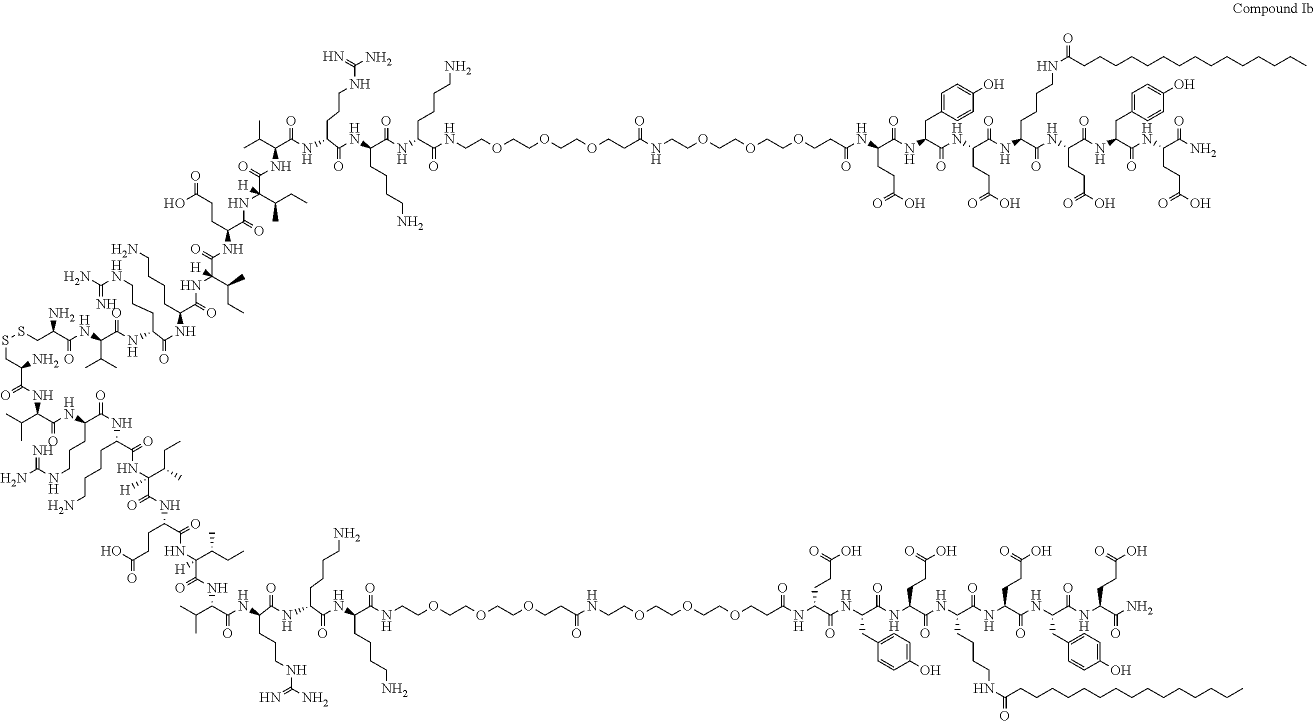

8. A method of preparing a compound of formula (I) or a salt thereof of any one of claims 1 to 4 wherein X═H, the method comprising:
(i) Preparing a PDGF Loop III monomer (Compound II) of the structure: 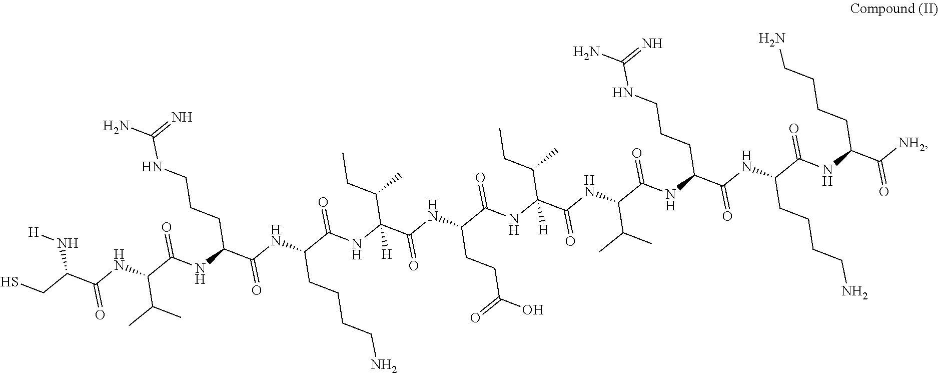 [SMILES: CC[C@H](C)[C@H](NC(=O)[C@H](CCCCN)NC(=O)[C@H](CCCNC(=N)N)NC(=O)[C@@H](NC(=O)[C@@H](N)CS)C(C)C)C(=O)N[C@@H](CCC(=O)O)C(=O)N[C@H](C(=O)N[C@H](C(=O)N[C@@H](CCCNC(=N)N)C(=O)N[C@@H](CCCCN)C(=O)N[C@@H](CCCCN)C(N)=O)C(C)C)[C@@H](C)CC]
(ii) Preparing a linked PDGF Loop III HSA monomer of formula II of the structure: 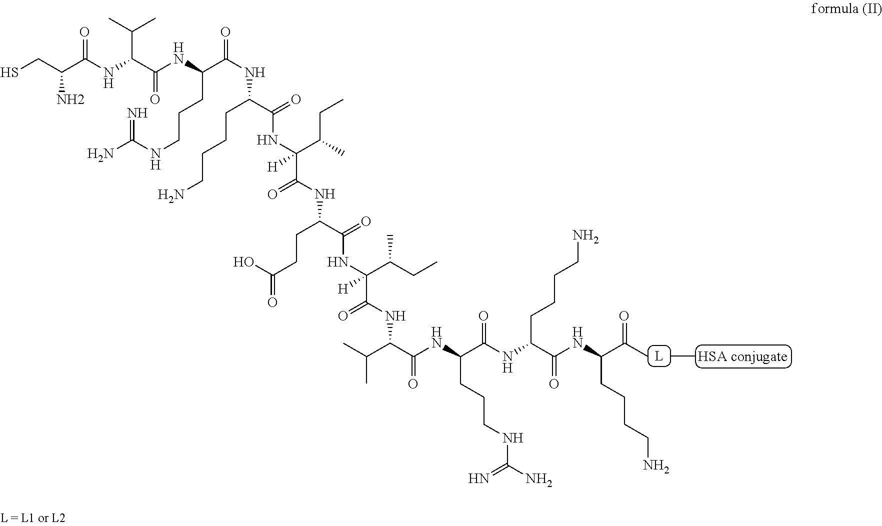
from the PDGF Loop III monomer (Compound II), and
(iii) Preparing a compound of formula (I) by disulfide coupling of the compound of formula (II) with the PDGF Loop III HSA monomer (Compound (II)) or with a second compound of formula (II).

9. The method of claim 8, wherein the PDGF Loop III monomer (Compound II) and/or the PDGF Loop III HSA monomer (formula II) are prepared by solid phase peptide synthesis.

10. A compound of formula (I) prepared by the method of claim 8 or claim 9.

11. A pharmaceutical composition comprising the compound of formula (I) or salt thereof of any one of claims 1 to 7 or claim 10 and a pharmaceutically acceptable excipient, carrier and/or diluent.

12. The pharmaceutical composition of claim 11, wherein the composition is formulated for parenteral administration.

13. A method of: reversing cardiac damage caused by myocardial infarct in a subject in need thereof, reversing cardiac damage in a subject suffering from chronic heart failure; treating cardiac damage caused by myocardial infarct in a subject in need thereof, treating cardiac damage in a subject suffering from chronic heart failure; restoring or improving cardiac function following myocardial infarct in a subject in need thereof, restoring or improving cardiac function in a subject suffering from chronic heart failure; enhancing cardiac repair following myocardial infarct in a subject in need thereof, enhancing cardiac repair in a subject suffering from chronic heart failure; treatment of cardiac dysfunction following myocardial infarction in a subject in need thereof; treatment of cardiac dysfunction in a subject suffering from chronic heart failure; treatment of persistent angina following myocardial infarction in a subject in need thereof, treatment of persistent angina in a subject suffering from chronic heart failure; treatment of persistent angina in a subject in need thereof, improving survival, decreasing arrhythmias, and/or increasing ventricular contraction and compliance following myocardial infarction in a subject in need thereof; improving survival, decreasing arrhythmias, and/or increasing ventricular contraction and compliance in a subject suffering from chronic heart failure; prolonging survival of a subject following myocardial infarction; prolonging survival of a subject suffering from chronic heart failure; preventing the development of severe heart failure in a subject following myocardial infarction; preventing the development of severe heart failure in a subject suffering from chronic heart failure; or of treatment of chronic heart failure in a subject in need thereof, the method comprising administering the compound of formula (I) or salt thereof of any one of claims 1 to 7 or claim 10, or the pharmaceutical composition of claim 11 or claim 12 to the subject.

14. The method of claim 13, wherein the compound of formula (I) or salt thereof is administered in an amount of between about 0.1 to 100 mg/kg; and/or wherein the compound of formula (I) is administered twice per day, once per day, twice per week, once per week, once every 2 weeks, monthly, once every two months, or once every 6 months; and/or wherein the duration of treatment is 1 day, 2 days, 3 days, 4 days, 5 days, 6 days, 7 days, 14 days, one month, two months, three months, four months, five months, six months, seven months, eight months, nine months, ten months, eleven months, or twelve months; and/or wherein administration of the compound of formula (I) of any one of claims 1 to 7 or claim 10, or the pharmaceutical composition of claim 11 or claim 12 to the subject does not significantly change the size of cardiac fibrosis post infarct.

15. The method of either claim 13 or claim 14, wherein administration of the compound of formula (I) of any one of claims 1 to 7 or claim 10, or the pharmaceutical composition of claim 11 or claim 12 to the subject does not significantly increase cardiac fibrosis post infarct compared to administration of a corresponding dosage of a homodimer of the platelet derived growth factor (PDGF)—B chain which is not conjugated to human serum albumin (HSA).
